(19) United States
(12) Patent Application Publication
(10) Pub. No.: US 2008/0253966 A1
(43) Pub. Date: Oct. 16, 2008

(54) Diagnostic, Prognostic, and Therapeutic Factor Smac/Diablo in Human Cancer

(75) Inventors: Benjamin Bonavida, Los Angeles, CA (US); Tsuneharu Miki, Sakyo-ku (JP); Yoichi Mizutani, Sakyo-ku (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of The University of California, Oakland, CA (US)

(21) Appl. No.: 11/667,514

(22) PCT Filed: Nov. 18, 2005
(86) PCT No.: PCT/US05/41896

§ 371 Date: Nov. 6, 2007

Related U.S. Application Data
(60) Provisional application No. 60/629,650, filed on Nov. 19, 2004.

(51) Int. Cl. A61K 49/00; G01N 33/574; C12Q 1/02; C07H 21/04; A61K 31/70; A61P 43/00

(52) U.S. Cl. 424/9.1; 435/7.23; 435/6; 536/24.31; 514/44

(57) ABSTRACT

The present invention provides, for the first time, the finding that Smac/DIABLO is underexpressed in cancers such as renal cell carcinoma. In particular, the present invention provides methods of diagnosing and providing a prognosis for cancers that underexpress Smac/DIABLO, as well as methods of drug discovery to identify therapeutics useful when used alone or in combination with other cancer therapeutics. The present invention also provides methods of treating or inhibiting cancers that underexpresses Smac/DIABLO, in which potentiation of Smac/DIABLO expression and/or activity sensitizes resistant tumor cells to cytotoxic treatments including chemotherapy, radiation therapy, hormonal therapy, and immunotherapy. Compositions, kits, and integrated systems for carrying out the diagnostic, prognostic, and therapeutic methods of the present invention are also provided.

### CROSS-REFERENCE TO RELATED APPLICATION

This application claims the benefit of U.S. Provisional Patent Application No. 60/629,650, filed Nov. 19, 2004, the content of which is hereby incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with Government support under Grant No. DAMD17-02-1-0023, awarded by the US Department of Defense. The US Government has certain rights in this invention.

### BACKGROUND OF THE INVENTION

Cancer is the second leading cause of death behind heart disease. In fact, cancer incidence and death figures account for about 10% of the U.S. population in certain areas of the United States (National Cancer Institute's Surveillance, Epidemiology, and End Results (SEER) database and Bureau of the Census statistics; see, Harrison's Principles of Internal Medicine, Kasper et al., 16th ed., 2005, Chapter 66). The five leading causes of cancer deaths among men are lung cancer, prostate cancer, colon and rectum cancer, pancreatic cancer, and leukemia. The five leading causes of cancer deaths among women are lung cancer, breast cancer, colon cancer, ovarian cancer, and pancreatic cancer. When detected at locally advanced or metastatic stages, no consistently curative treatment regimen exists. Treatment for metastatic cancer includes immunotherapy, hormonal ablation, radiation therapy, chemotherapy, hormonal therapy, and combination therapies. Unfortunately, for prostate cancer and hormone dependent tumors, there is frequent relapse of an aggressive androgen independent disease that is insensitive to further hormonal manipulation or to treatment with conventional chemotherapy (Ghosh et al., Proc. Natl. Acad. Sci. USA, 95:13182-13187 (1998)). Therefore, there is a need for alternative therapies, such as immunotherapy or reversal of resistance to chemotherapy, radiation therapy, and hormonal therapy. For instance, immunotherapy is predicated on the notion that all drug-resistant tumors should succumb to cytotoxic lymphocyte-mediated killing. Such tumors may also develop cross-resistance to apoptosis-mediated cytotoxic lymphocytes, resulting ultimately in tumor progression and metastasis of the resistant cells (Thompson, Science, 267:1456-1462 (1995)). The mechanism responsible for the anti-apoptotic phenotype may be useful as a prognostic and/or diagnostic indicator and target for immunotherapeutic intervention or reversal of resistance to other cytotoxic therapies.

Cell death by apoptosis occurs when the intracellular apoptotic pathway is activated (Vaux et al., Proc. Natl. Acad. Sci. USA, 93:2239-2244 (1996)). Signals inducing apoptosis can be very diverse and encompass the direct stimulation of death receptors or cellular stress induced by chemicals and irradiation. The ability to evade apoptosis may enhance the cells' propensity to malignancy. The classical apoptotic pathway consists of activation of the caspase family cascade. The effector caspase 3 serves to cleave cellular protein substrates and brings about the apoptotic phenotype. Caspase 3 can be either activated by caspase 8 or by a signaling complex referred to as the apoptosome, consisting of cytochrome c, Apaf-1, and caspase 9. Cytochrome c allows the oligomerization of Apaf-1, thereby activating caspase 9 in the process. X-linked inhibitor of apoptosis protein (XIAP) has the potential to inhibit active caspase 3 and slows down the process at this step (Bratton et al., EMBO J, 20:998-1009, (2001)).

Apoptogenic factors that are normally sequestered in the mitochondria are released into the cytosol during the mitochondria-dependent pathway for apoptosis. These factors include second mitochondria-derived activator of caspase/direct inhibitor of apoptosis-binding protein with low pI (Smac/DIABLO), endonuclease G, cytochrome c, and Omni/HtrA2 (van Gurp et al., Biochem. Biophys. Res. Commun., 304:487-497 (2003)). The release of cytochrome c into the cytoplasm is not always sufficient to initiate the caspase cascade. Endogenous inhibitors of apoptosis proteins (IAPs) including XIAP are present and, thus, prevent the activation of pro-caspases. Therefore, the inhibition of the activation of pro-caspases interferes with the activation of mature caspases. Murine Smac and its human ortholog DIABLO are 29 kD mitochondria precursor proteins proteolytically cleaved in the mitochondria to a 23 kD mature form and released into the cytosol after an apoptotic stimulus (Du et al., Cell, 102:33-42 (2000); Verhagen et al., Cell, 102:33-42 (2000)). Smac/DIABLO acts as a dimer and contributes to caspase activation by sequestering IAPs (Srinivasula et al., J. Biol. Chem., 275:36152-36157 (2000)).

Recent studies have reported that overexpression of Smac/DIABLO can induce apoptosis and/or sensitize the resistant cancer cells to death receptor- or cytotoxic drug-induced apoptosis (Fulda et al., Nature Med., 8:808-815 (2002); Ng et al., Mol. Cancer. Ther., 1:1051-1058, (2002)). These findings suggest that Smac/DIABLO plays an important role in the regulation of apoptotic responses in cancer cells to both immune- and drug-mediated therapies. The association between the levels of Smac/DIABLO expression and tumor cell progression, however, is not known. In fact, only one study has examined the expression of Smac/DIABLO in cancer (Yoo et al., APMIS, 111:382-388 (2003)). As a result, there is a need in the art for a better understanding of the role of Smac/DIABLO in tumor progression and therapy-resistant cancers. There is also a need in the art for methods of diagnosing or providing a prognosis for cancers such as renal cell carcinoma (RCC) based upon the levels of Smac/DIABLO expression. The present invention satisfies these and other needs.

### BRIEF SUMMARY OF THE INVENTION

The present invention provides, for the first time, the finding that Smac/DIABLO is underexpressed in cancers such as RCC, and therefore has clinical significance as a diagnostic and/or prognostic marker, as well as a target for drug development. The present invention also provides methods of treating or inhibiting cancers that underexpresses Smac/DIABLO (e.g., therapy resistant cancers) by administering a therapeutically effective amount of one or more Smac/DIABLO modulators (e.g., mimetics or agonists). Compositions, kits, and integrated systems for carrying out the diagnostic, prognostic, and therapeutic methods of the present invention are also provided.

In one aspect, the present invention provides a method of diagnosing a cancer that underexpresses Smac/DIABLO, the method comprising the steps of:
(a) contacting a tissue sample with an antibody that specifically binds to Smac/DIABLO protein; and
(b) determining whether or not Smac/DIABLO protein is underexpressed in the sample, thereby diagnosing the cancer that underexpresses Smac/DIABLO.

In another aspect, the present invention provides a method of diagnosing a cancer that underexpresses Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid in the sample is underexpressed in the sample, thereby diagnosing the cancer that underexpresses Smac/DIABLO.

In yet another aspect, the present invention provides a method of providing a prognosis for a cancer that underexpresses Smac/DIABLO, the method comprising the steps of:
(a) contacting a tissue sample with an antibody that specifically binds to Smac/DIABLO protein; and
(b) determining whether or not Smac/DIABLO protein is underexpressed in the sample, thereby providing a prognosis for the cancer that underexpresses Smac/DIABLO.

In still yet another aspect, the present invention provides a method of providing a prognosis for a cancer that underexpresses Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid is underexpressed in the sample, thereby providing a prognosis for the cancer that underexpresses Smac/DIABLO.

In one embodiment, circulating levels of Smac/DIABLO can be detected for prognostic and diagnostic uses. Detection of the pro-form vs. activated form of the protein and localization of the pro-form and the activated form can also be used prognostically and diagnostically.

Generally, the methods find particular use in diagnosing or providing a prognosis for cancer including renal cancer (i.e., renal cell carcinoma), bladder cancer, prostate cancer, lung cancer, ovarian cancer, breast cancer, colon cancer, leukemias, B-cell lymphomas (e.g., non-Hodgkin's lymphomas, including Burkitt's, Small Cell, and Large Cell lymphomas), hepatocarcinoma, or multiple myeloma.

The present invention also provides an isolated primer set, the primer set comprising a first oligonucleotide and a second oligonucleotide, each oligonucleotide comprising a nucleotide sequence of about 50 nucleotides or less, wherein the first oligonucleotide comprises SEQ ID NO: 1 and the second oligonucleotide comprises SEQ ID NO:2.

In addition, the present invention provides a method of localizing a cancer that underexpresses Smac/DIABLO in vivo, the method comprising the step of imaging in a subject a cell underexpressing Smac/DIABLO (e.g., protein and/or RNA), thereby localizing the cancer in vivo.

The present invention further provides a method of identifying a compound that inhibits a cancer that underexpresses Smac/DIABLO or a therapy resistant cancer, the method comprising the steps of:
(a) contacting a cell expressing Smac/DIABLO with a compound; and
(b) determining the effect of the compound on Smac/DIABLO expression, thereby identifying a compound that inhibits the cancer that underexpresses Smac/DIABLO or the therapy resistant cancer.

The methods of screening find particular use in identifying compounds that modulate (i.e., increase) Smac/DIABLO protein and/or RNA expression/activity in cancers such as renal cancer (i.e, renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, or multiple myeloma.

The present invention also provides a method of treating or inhibiting a cancer that underexpresses Smac/DIABLO or a therapy resistant cancer in a subject comprising administering to the subject a therapeutically effective amount of one or more Smac/DIABLO mimetics (e.g., agent that binds one or more IAPs) or agonists (e.g., nucleic acid encoding Smac/DIABLO for gene therapy).

The Smac/DIABLO mimetics or agonists can be administered alone or co-administered (e.g., concurrently or sequentially) in combination therapy with conventionally used chemotherapy, radiation therapy, hormonal therapy, and/or immunotherapy. The methods find particular use in treating renal cancer (i.e., renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, multiple myeloma, or other cancers that underexpress Smac/DIABLO or have Smac/DIABLO-associated resistance to apoptotic-induced stimuli.

Other objects, features, and advantages of the present invention will be apparent to one of skill in the art from the following detailed description and figures.

### BRIEF DESCRIPTION OF THE DRAWINGS

FIG. 1 shows the expression of Smac/DIABLO in RCC cell lines (FIG. 1A) and the expression of Smac/DIABLO in RCC and the normal kidney (FIG. 1B). N: Normal kidney; T: RCC.

FIG. 2 shows the expression of Smac/DIABLO in RCC and the normal kidney for Cases 2-11 (FIGS. 2A and 2B) and the expression of Smac/DIABLO in primary and metastatic RCC for Case 12 (brain metastasis), Case 13 (bone metastasis), and Case 14 (bone metastasis) (FIG. 2C). N: Normal kidney; T: RCC; PT: Primary RCC; MT: Metastatic RCC.

FIG. 3 shows the expression of Smac/DIABLO in oncocytoma. N: Normal kidney, T: Oncocytoma.

FIG. 4 shows the relationship between Smac/DIABLO expression and postoperative disease-specific survival in patients with RCC. Solid line: 64 patients with positive Smac/DIABLO expression. Dashed line: 14 patients with negative Smac/DIABLO expression.

### DETAILED DESCRIPTION OF THE INVENTION

### I. Introduction

Renal cell carcinoma (RCC) accounts for about 2% of all cancer cases worldwide (Motzer et al., N. Engl. J. Med., 335:865-875 (1996)). Metastatic disease is often present at the time of diagnosis of RCC, and its poor response to chemotherapy and radiotherapy determines its poor prognosis.

The present invention is based, in part, on the surprising discovery that underexpression of Smac/DIABLO in RCC as well as other cancers (e.g., bladder cancer) can be used as a diagnostic and/or prognostic marker, and that enhancement of Smac/DIABLO expression in RCC as well as other cancers can potentiate the effect of other cancer therapies (e.g., immunotherapy, chemotherapy, radiotherapy, hormonal therapy, etc.). In particular, the present invention demonstrates for the first time that Smac/DIABLO expression is downregulated in RCC and that no Smac/DIABLO expression in RCC predicted a worse prognosis. In addition, the present invention illustrates that transfection with Smac/DIABLO sensitized RCC to TRAIL/cisplatin-induced apoptosis. Thus, the present invention shows the diagnostic and prognostic significance of Smac/DIABLO for RCC and other cancers. Smac/DIABLO gene family members with the same function will also serve as important diagnostic, prognostic, and therapeutic targets.

Smac-Diablo is produced as a precursor protein that contains an MTS (mitochondrial targeting sequence) that remains non-apoptotic. The pro-apoptotic activity of Smac-Diablo is obtained when its MTS is cleaved after been transported to the mitochondria. The pro form is cleaved in the mitochondria, and it is the cleaved form that is released from the activated mitochondria following an apoptotic stimulus. After it is released, it inhibits the IAP's and the ratio of Smac-Diablo IAP's will dictate the activation of caspase 3 and apoptosis. The five amino acids (A, V, P, I, A) at the amino terminal, which is exposed after cleavage of the MTS, is thought to be responsible for the interaction with the IAP's and therefore inhibiting their functions. Therefore, the Smac-Diablo expression as well as its localization and function (pro-form vs. active, cleaved form) are useful for diagnostic, prognostic and therapeutic applications. The localization of Smac-Diablo in the nucleus, the mitochondria, and the cytoplasm correlates with resistance and is also a predictor of therapeutic outcome. Smac-Diablo levels in circulating blood are also a predictor of therapeutic outcome and can be used as a convenient diagnostic and prognostic assay for therapy resistance and outcome. One can examine relative amounts of pro-form vs. cleaved form, activity of the cleaved form, expression of Smac-Diablo (nucleic acid and protein), stability of RNA and protein, splicing, etc. for diagnostics and prognostics. Pro-form and active form are both useful as therapeutic targets for drug assays. One can use Smac-Diablo to assay for specific agents that act to promote Smac-Diablo transcription, RNA processing and splicing, translation, protein processing of pro-form to active form, protein stability, protein activity, and protein localization.

Detection of Smac/DIABLO expression is particularly useful as a diagnostic and/or prognostic indicator for cancers such as renal cancer (i.e., renal cell carcinoma), bladder cancer, prostate cancer, lung cancer, ovarian cancer, breast cancer, colon cancer, leukemias, B-cell lymphomas (e.g., non-Hodgkin's lymphomas, including Burkitt's, Small Cell, and Large Cell lymphomas), hepatocarcinoma, and multiple myeloma. Detection can include, for example, the level of Smac/DIABLO mRNA or protein expression, or the localization (e.g., nuclear, cyoplasmic, mitochondrial, etc.) of Smac/DIABLO mRNA or protein. Expression of DIABLO can be examined in whole cell or tissue samples. In terms of early diagnosis, needle, surgical, or bone marrow biopsies can be used and examined by techniques such as immunoblotting or immunohistochemistry and compared to control cells or tissue, e.g., from a healthy subject. In addition, microlaser microdissection can be used to isolate a few cells and run RT-PCR for Smac/DIABLO nucleic acid. The following PCR primers can be used to detect Smac/DIABLO nucleic acid: (sense, SEQ ID NO:1) 5′CGCGGATCCATGGCGGCTCTGAAGAGTTG 3′; and (antisense, SEQ ID NO:2) 5′GCTCTCTAGACTCAGGCCCTCAATCCTCA 3′. Molecular imaging can be used to identify individual cells or groups of cells that express specific proteins or enzymatic activity in real time in living patients (Louie et al., 2002). The ability to image Smac/DIABLO can provide the localization of cancers within the tissue of a primary tumor and tissues of metastatic tumors. One application of this technique is to help direct the location of needle biopsy sites in the kidney and to assess the extent of cancer within the kidney. In addition, the ability to image Smac/DIABLO can systematically provide value for the detection of metastatic RCC and cancers in other organs such as the bladder. In addition to altered (e.g., lowered or absent) expression of Smac/DIABLO in cancers, e.g., RCC, the same effects can be seen in cells with functional mutations in Smac/DIABLO, such as loss of activity, loss of cleavage site, failure to transport to and from the mitochondria, etc.

Overexpression of Smac/DIABLO can sensitize tumor cells to both chemotherapy and immunotherapy. This result indicates a reversal of resistance by agents (e.g., mimetics, agonists, etc.) that can either mimic Smac/DIABLO or upregulate its expression. Therefore, cells expressing Smac/DIABLO can be used for drug discovery to identify new drugs to treat RCC and other cancers, as well as to evaluate immunotherapeutic and chemotherapeutic cancer treatments. In addition, mitochondria expressing Smac/DIABLO can be used to assay for therapeutics. Drugs of particular interest would be capable of mimicking the action of Smac/DIABLO or upregulating Smac/DIABLO expression or function (e.g., small organic molecules, plasmids, RNAi, sense and antisense oligonucleotides, peptides, inhibitors of the proteasome, inhibitors of ubiquitination, etc.). Such drugs can be directly used alone or in combination with chemotherapy, radiotherapy, hormonal therapy, and/or immunotherapy to treat RCC as well as other cancers that are resistant to such therapy. Such drugs can also be used to slow or halt tumor progression and metastasis. Finally, tumor cell response to therapy can be improved by enhancing Smac/DIABLO expression. Based on changes in expression patterns, one can tailor specific therapies to cancer patients.

Accordingly, in a first aspect, the present invention provides a method of diagnosing a cancer that underexpresses Smac/DIABLO in a subject, e.g., by detecting underexpression of Smac/DIABLO, the method comprising the steps of:
(a) contacting a tissue sample from the subject with an antibody that specifically binds to Smac/DIABLO protein; and
(b) determining whether or not Smac/DIABLO protein is underexpressed in the sample, thereby diagnosing the cancer that underexpresses Smac/DIABLO. The antibody can be a monoclonal antibody or a polyclonal antibody, but is typically a monoclonal antibody.

In another aspect, the present invention provides a method of diagnosing a cancer that underexpresses Smac/DIABLO, e.g., by detecting underexpression of Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid in the sample is underexpressed in the sample, thereby diagnosing the cancer that underexpresses Smac/DIABLO. In one embodiment, the first oligonucleotide comprises SEQ ID NO:1 and the second oligonucleotide comprises SEQ ID NO:2.

In yet another aspect, the present invention provides a method of providing a prognosis for a cancer that underexpresses Smac/DIABLO, e.g., by detecting underexpression of Smac/DIABLO, the method comprising the steps of:
(a) contacting a tissue sample with an antibody that specifically binds to Smac/DIABLO protein; and
(b) determining whether or not Smac/DIABLO protein is underexpressed in the sample, thereby providing a prognosis for the cancer that underexpresses Smac/DIABLO. The antibody can be a monoclonal antibody or a polyclonal antibody, but is typically a monoclonal antibody.

In still yet another aspect, the present invention provides a method of providing a prognosis for a cancer that underexpresses Smac/DIABLO, e.g., by detecting underexpression of Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid is underexpressed in the sample, thereby providing a prognosis for the cancer that underexpresses Smac/DIABLO. In one embodiment, the first oligonucleotide comprises SEQ ID NO:1 and the second oligonucleotide comprises SEQ ID NO:2.

The diagnostic and prognostic methods of the present invention can also be carried out by determining the extent of Smac/DIABLO protein from a subject that binds to one or more inhibitor of apoptosis protein (IAP) family members, wherein decreased binding relative to a healthy subject indicates a cancerous phenotype. The diagnosis and prognosis methods can also be carried out by determining whether or not Smac/DIABLO protein is localized in the mitochondria or the cytosol of a cell, wherein Smac/DIABLO localization in the mitochondria, e.g., after an apoptotic stimulus, indicates a cancerous phenotype. The diagnosis and prognosis methods can also be carried out by determining whether or not Smac/DIABLO protein is full-length or truncated.

In determining the levels of protein expression or the localization of Smac/DIABLO protein, polyclonal or monoclonal antibodies that specifically bind Smac/DIABLO can be used.

Generally, the methods of the present invention find particular use in diagnosing or providing a prognosis for renal cancer (i.e., renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, or multiple myeloma. Preferably, the methods of the present invention are used in diagnosing or providing a prognosis for renal cell carcinoma (RCC) or a subtype thereof, e.g., clear-cell RCC, papillary RCC, Bellini duct carcinoma, chromophobe RCC, or renal oncocytoma. In carrying out the diagnostic or prognostic methods described herein, the determination of whether or not Smac/DIABLO is underexpressed can be made, e.g., by comparing a test biological sample to a control autologous biological sample from normal tissue.

In certain instances, the methods of diagnosis or prognosis are carried out by determining the extent by which Smac/DIABLO protein from test tissue binds to an IAP family member compared to Smac/DIABLO from normal tissue, for example, by employing an in vitro binding assay.

In carrying out the diagnostic or prognostic methods of the present invention, the tissue sample can be taken from a tissue of a primary tumor or a metastatic tumor. A tissue sample can be taken, for example, by an excisional biopsy, an incisional biopsy, a needle biopsy, a surgical biopsy, a bone marrow biopsy, or any other biopsy technique known in the art. In some embodiments, the tissue sample is microlaser microdissected cells from a needle biopsy. In other embodiments, the tissue sample is a metastatic cancer tissue sample. In yet other embodiments, the tissue sample is fixed, e.g., with paraformaldehyde, and embedded, e.g., in paraffin. Suitable tissue samples can be obtained from cancers such as kidney, bladder, prostate, ovary, lung, colon, breast, etc., as well as from the blood, serum, saliva, urine, bone, lymph node, liver, or tissue.

In another aspect, the present invention also provides an isolated primer set, the primer set comprising a first oligonucleotide and a second oligonucleotide, each oligonucleotide comprising a nucleotide sequence of about 50 nucleotides or less (e.g., about 50, 45, 40, 35, 30, 25, 20, 15, or 10 nucleotides or less), wherein the first oligonucleotide comprises SEQ ID NO:1 and the second oligonucleotide comprises SEQ ID NO:2.

In addition, the present invention provides a method of localizing a cancer that underexpresses Smac/DIABLO in vivo, the method comprising the step of imaging in a subject a cell underexpressing Smac/DIABLO (e.g., protein and/or RNA), thereby localizing the cancer in vivo.

The present invention also provides a method of identifying a compound that inhibits a cancer that underexpresses Smac/DIABLO, the method comprising the steps of:
(a) contacting a cell expressing Smac/DIABLO with a compound; and
(b) determining the effect of the compound on Smac/DIABLO expression, thereby identifying a compound that inhibits the cancer that underexpresses Smac/DIABLO.

The present invention further provides a method of identifying a compound that inhibits a therapy resistant cancer, the method comprising the steps of:
(a) contacting a cell expressing Smac/DIABLO with a compound; and
(b) determining the effect of the compound on Smac/DIABLO expression, thereby identifying a compound that inhibits the therapy resistant cancer.

The methods of screening find particular use in identifying compounds that modulate (i.e., increase) Smac/DIABLO protein and/or RNA expression/activity in cancers such as renal cancer (i.e, renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, or multiple myeloma.

In carrying out the methods of screening, the compound can be, for example, a small organic molecule, a chemical inhibitor, a polypeptide, an antibody, a polynucleotide (e.g., plasmid). In some embodiments, the compound induces or increases Smac/DIABLO expression, for example, transcription and/or translation, RNA processing, RNA and protein stability, localization, protein processing, and protein activity. In certain instances, the compound promotes Smac/DIABLO transcription by activating transcription factors. In certain other instances, the compound promotes Smac/DIABLO function by increasing the binding affinity of Smac/DIABLO for one or more IAP family members. In other embodiments, the compound sensitizes a cell to apoptosis induced by cell signaling through a death receptor (e.g., Fas ligand receptor, TRAIL receptor, TNF-R1, etc.) or through conventional cytotoxic therapies. In additional embodiments, the compound directly or indirectly has an effect on Smac/DIABLO mRNA, e.g., by inhibiting its degradation, by increasing its stability, by facilitating its translation, etc. In further embodiments, the compound directly or indirectly has an effect on Smac/DIABLO protein, e.g., by inhibiting its degradation, by increasing its stability, by facilitating its maturation, etc. As a non-limiting example, the compound can slow the degradation of Smac/DIABLO via the proteasome system.

Typically, the compound will inhibit a cancer that underexpresses Smac/DIABLO or a therapy resistant cancer in combination with another cancer treatment, for example, co-administration (concurrently or sequentially) with a death receptor agonist or another chemotherapeutic agent known in the art. Compounds of interest that increase Smac/DIABLO expression and/or activity can sensitize cancer cells to conventional cancer treatments, including chemotherapy, radiotherapy, hormonal therapy, immunotherapy, and other methods of treating therapy resistant cancer, alone or in combination.

The present invention also provides a method of treating or inhibiting a cancer that underexpresses Smac/DIABLO or a therapy resistant cancer in a subject comprising administering to the subject a therapeutically effective amount of one or more Smac/DIABLO mimetics or agonists.

In another aspect, the present invention provides a method of sensitizing a tumor to conventional cancer treatment (e.g., chemotherapy, radiation therapy, hormonal therapy, and immunotherapy) comprising administering to the subject a therapeutically effective amount of one or more Smac/DIABLO mimetics or agonists.

The Smac/DIABLO mimetic or agonist can be a known compound (see, e.g., Sun et al., J. Med. Chem., 47:4147-4150 (2004)), a polynucleotide sequence (e.g., a plasmid encoding Smac/DIABLO), an inhibitory RNA sequence (e.g., a Smac/DIABLO siRNA or antisense RNA), an antibody, or combinations thereof. The Smac/DIABLO mimetic or agonist can also be identified according to the screening methods of the present invention.

In carrying out the methods of treatment, the one or more Smac/DIABLO mimetics or agonists can be administered concurrently or sequentially with conventional therapies, for example, currently used chemotherapy, radiation therapy, hormonal therapy, or immunotherapy treatments. In one embodiment, the Smac/DIABLO mimetic or agonist is co-administered with a second pharmacological agent, for example, an agonist of a death receptor, including a Fas ligand receptor (e.g., Fas), a TRAIL receptor (e.g., DR4 or DR5), or TNF-R1. The death receptor agonist can be an antibody, including a monoclonal antibody or a polyclonal antibody. In certain instances, the Smac/DIABLO mimetic or agonist is co-administered with a monoclonal antibody against a DR5 receptor. In certain other instances, the Smac/DIABLO mimetic or agonist is co-administered with a TRAIL polypeptide.

The one or more Smac/DIABLO mimetics or agonists can be co-administered simultaneously or sequentially with another therapeutic agent. In one embodiment, one or more Smac/DIABLO mimetics or agonists are administered prior to administering another therapeutic agent. This strategy can establish a sensitizing effect on the cell before administering a cytotoxic agent. In other embodiments, one or more Smac/DIABLO mimetics or agonists are administered concurrently with another therapeutic agent or after administering another therapeutic agent.

As a non-limiting example, the Smac/DIABLO mimetic or agonist can be co-administered with conventional chemotherapeutic agents including alkylating agents (e.g., cisplatin, cyclophosphamide, carboplatin, ifosfamide, chlorambucil, busulfan, thiotepa, nitrosoureas, etc.), anti-metabolites (e.g., 5-fluorouracil, azathioprine, methotrexate, fludarabine, etc.), plant alkaloids (e.g., vincristine, vinblastine, vinorelbine, vindesine, podophyllotoxin, paclitaxel, docetaxel, etc.), topoisomerase inhibitors (e.g., amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, plicamycin, etc.), and the like. The Smac/DIABLO mimetic or agonist can also be co-administered with conventional hormonal therapeutic agents including, but not limited to, steroids (e.g., dexamethasone), finasteride, aromatase inhibitors, tamoxifen, and gonadotropin-releasing hormone agonists (GnRH) such as goserelin. Additionally, the Smac/DIABLO mimetic or agonist can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.). In a further embodiment, the Smac/DIABLO mimetic or agonist can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰_{Y}, ¹⁰⁵Rh, Ag, ¹¹¹In, ^{117m}Sn, ¹⁴⁹ Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In preferred embodiments, the Smac/DIABLO mimetic and agonist is an agent that targets one or more IAP family members and a nucleic acid (e.g., plasmid) encoding Smac/DIABLO for gene therapy, respectively. The therapeutic methods described herein find particular use in treating renal cancer (i.e., renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, multiple myeloma, or other cancers that underexpress Smac/DIABLO or have Smac/DIABLO-associated resistance to apoptotic-induced stimuli.

### II. Definitions

As used herein, the following terms have the meanings ascribed to them unless specified otherwise.

“DIABLO” or “Smac/DIABLO” refers to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, e.g., about 65%, 70%, 75%, 80%, 85%, 90%, 95%, preferably about 91%, 92%, 93%, 94%, 95%, 96%, 97%, 98%, 99%, or greater amino acid sequence identity, preferably over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; and/or (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate (e.g., human), rodent (e.g., rat, mouse, hamster), cow, pig, horse, sheep, or any mammal. The nucleic acids and proteins of the present invention include both naturally-occurring and recombinant molecules. Smac typically refers to the mouse ortholog and DIABLO typically refers to the human ortholog. Exemplary human genes for DIABLO are provided by Accession Nos. AF298770, BC004417, and NM_{—}138930; exemplary protein sequences are provided by Accession Nos. AAG22077, AAH04417, and NP_{—}620308. Truncated, alternatively spliced, precursor, and mature forms of Smac/DIABLO are also included in the foregoing definition.

The term “cancer” refers to human cancers and carcinomas, sarcomas, adenocarcinomas, lymphomas, leukemias, solid and lymphoid cancers, etc. Examples of different types of cancer include, but are not limited to, renal cancer (i.e., renal cell carcinoma), bladder cancer, lung cancer, breast cancer, thyroid cancer, liver cancer (i.e., hepatocarcinoma), pleural cancer, pancreatic cancer, ovarian cancer, uterine cancer, cervical cancer, prostate cancer, testicular cancer, colon cancer, anal cancer, pancreatic cancer, bile duct cancer, gastrointestinal carcinoid tumors, esophageal cancer, gall bladder cancer, rectal cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. In preferred embodiments, the methods of the present invention are useful for diagnosing, proving a prognosis for, and treating renal cell carcinoma (RCC) or a subtype thereof, e.g., clear-cell RCC, papillary RCC, Bellini duct carcinoma, chromophobe RCC, or renal oncocytoma.

“Therapy resistant” cancers, tumor cells, and tumors refer to cancers that have become resistant to both apoptosis-mediated (e.g., through death receptor dell signaling, for example, Fas ligand receptor, TRAIL receptors, TNF-R1, chemotherapeutic drugs, radiation, etc.) and non-apoptosis mediated (e.g., toxic drugs, chemicals, etc.) cancer therapies including, but not limited to, chemotherapy, hormonal therapy, radiotherapy, immunotherapy, and combinations thereof.

“Therapeutic treatment” and “cancer therapies” refers to apoptosis-mediated and non-apoptosis mediated cancer therapies including, without limitation, chemotherapy, hormonal therapy, radiotherapy, immunotherapy, and combinations thereof. Cancer therapies can be enhanced by co-administration with a sensitizing agent, such as a Smac/DIABLO mimetic (e.g., for inhibiting one or more inhibitor of apoptosis proteins (IAPs)) or a Smac/DIABLO agonist (e.g., a nucleic acid for gene therapy).

The terms “underexpress,” “underexpression,” or “underexpressed” interchangeably refer to a gene that is transcribed or translated at a detectably lower level, usually in a cancer cell or tissue, in comparison to a normal cell or tissue. Underexpression therefore refers to both underexpression of Smac/DIABLO protein (both pro-form and active, processed form) and RNA (e.g., due to decreased transcription, post-transcriptional processing, translation, post-translational processing, altered stability, altered protein degradation, etc.), as well as local underexpression due to altered protein trafficking patterns (e.g., decreased cellular or subcellular localization), and/or reduced functional activity (e.g., as an IAP binding/inhibitory factor). Underexpression can be detected using conventional techniques for detecting protein (i.e., ELISA, Western blotting, flow cytometry, immunofluorescence, immunohistochemistry, DNA binding assays, etc.) or mRNA (e.g., RT-PCR, PCR, hybridization, etc.). One skilled in the art will know of other techniques suitable for detecting underexpression of Smac/DIABLO protein or mRNA. Underexpression of Smac/DIABLO can be at least about 5%, 10%, 15%, 20%, 25%, 30%, 35%, 40%, 45%, 50%, 55%, 60%, 65%, 70%, 75%, 80%, 85%, 90%, or 95% in comparison to a normal cell. In certain instances, underexpression of Smac/DIABLO comprises at least about a 1-fold, 2-fold, 3-fold, 4-fold, 5-fold, 6-fold, or 7-fold lower level of transcription or translation in comparison to a normal cell. Underexpression further includes no expression, i.e., expression that is undetectable or insignificant.

The term “cancer that underexpresses Smac/DIABLO” refers to cancer cells or tissues that underexpress Smac/DIABLO, in accordance with the above definition. This term also encompasses Smac/DIABLO-mediated resistance to apoptosis through death receptors (e.g., TNF-R1, Fas ligand receptors, TRAIL receptors, etc.), optionally in combination with the administration of chemotherapeutic drugs, radiation therapy, immunotherapy, and/or hormonal therapy.

The terms “cancer-associated antigen,” “tumor-specific marker,” or “tumor marker” interchangeably refers to a molecule (typically protein, carbohydrate, or lipid) that is preferentially expressed in a cancer cell in comparison to a normal cell, and which is useful for the preferential targeting of a pharmacological agent to the cancer cell. A marker or antigen can be expressed on the cell surface or intracellularly. Oftentimes, a cancer-associated antigen is a molecule that is overexpressed or stabilized with minimal degradation in a cancer cell in comparison to a normal cell, for instance, 1-fold over expression, 2-fold overexpression, 3-fold overexpression, or more in comparison to a normal cell. Oftentimes, a cancer-associated antigen is a molecule that is inappropriately synthesized in the cancer cell, for instance, a molecule that contains deletions, additions, or mutations in comparison to the molecule expressed on a normal cell. Oftentimes, a cancer-associated antigen will be expressed exclusively in a cancer cell and not synthesized or expressed in a normal cell. Exemplified cell surface tumor markers include the proteins c-erbB-2 and human epidermal growth factor receptor (HER) for breast cancer, PSMA for prostate cancer, and carbohydrate mucins in numerous cancers, including breast, ovarian, and colorectal. Exemplified intracellular tumor markers include, for example, mutated tumor suppressor or cell cycle proteins, including p53.

The term “mimetic” refers to an agent that mimics the function or activity of a polypeptide of the present invention. Mimetics include naturally-occurring and synthetic proteins, polypeptides, oligopeptides, antibodies, small organic molecules, polysaccharides, lipids, fatty acids, polynucleotides, inhibitory RNA molecules, oligonucleotides, etc. Preferably, the mimetic is capable of mimicking the function or activity of Smac/DIABLO protein, e.g., by binding to and sequestering one or more inhibitor of apoptosis protein (IAP) family members (e.g., cellular inhibitor of apoptosis protein (cIAP), X-linked inhibitor of apoptosis protein (XIAP), etc.). Suitable Smac/DIABLO mimetics that can be used in the therapeutic methods of the present invention include, but are not limited to, the conformationally-constrained Smac/DIABLO mimetics that target the XIAP/caspase-9 interaction site as described in Sun et al., J. Med. Chem., 47:4147-4150 (2004).

As used herein, the term “agonist” refers to an agent that binds to a polypeptide or polynucleotide (e.g., DNA or RNA) of the present invention and stimulates, increases, activates, facilitates, enhances activation, sensitizes, or up-regulates the activity or expression of the polypeptide or polynucleotide. In certain instances, the agonist binds to a Smac/DIABLO polypeptide and affects the activity or expression of the polypeptide, e.g., by increasing its affinity for targets such as IAPs, by increasing its stability, by decreasing its degradation, by enhancing its proteolytic processing, by facilitating its transport out of the mitochondria, by increasing its post-translational processing, etc. In certain other instances, the agonist binds to Smac/DIABLO DNA and affects the activity or expression of the DNA, e.g., by increasing its transcription. In certain additional instances, the agonist binds to Smac/DIABLO RNA and affects the activity or expression of the RNA, e.g., by increasing its translation, by increasing its stability, by decreasing its degradation, by increasing its post-transcriptional processing, etc.

An “antagonist” refers to an agent that inhibits the activity or expression of a polypeptide or polynucleotide of the present invention or binds to, partially or totally blocks stimulation, decreases, prevents, delays activation, inactivates, desensitizes, or down regulates the activity of the polypeptide or polynucleotide.

“Inhibitors,” “activators,” and “modulators” of expression or activity are used to refer to inhibitory, activating, or modulating molecules, respectively, identified using in vitro and in vivo assays for expression or activity, e.g., ligands, mimetics, agonists, antagonists, and their homologs and derivatives. The term “modulator” includes inhibitors and activators. Inhibitors are agents that, e.g., inhibit expression, e.g., translation, post-translational processing, stability, degradation, or nuclear or cytoplasmic localization of a polypeptide, or transcription, post transcriptional processing, stability or degradation of a polynucleotide of the present invention. Inhibitors can also bind to, partially or totally block stimulation or activity, decrease, prevent, delay activation, inactivate, desensitize, or down-regulate the activity of a polypeptide or polynucleotide of the present invention, e.g., antagonists. Activators are agents that, e.g., induce or activate the expression of a polypeptide or polynucleotide of the present invention or bind to, stimulate, increase, open, activate, facilitate, enhance activation or activity, sensitize, or up-regulate the activity of a polypeptide or polynucleotide of the present invention, e.g., agonists. Modulators include naturally-occurring and synthetic ligands, mimetics, antagonists, agonists, small chemical molecules, antibodies, inhibitory RNA molecules (i.e., siRNA or antisense RNA), and the like. Assays to identify inhibitors and activators include, e.g., applying putative modulator compounds to cells, in the presence or absence of a polypeptide or polynucleotide of the present invention, and then determining the functional effects on polypeptide or polynucleotide activity. Samples or assays comprising a polypeptide or polynucleotide of the present invention that are treated with a potential activator, inhibitor, or modulator are compared to control samples without the inhibitor, activator, or modulator to examine the extent of effect. Control samples (untreated with modulators) can be assigned a relative activity value of 100%. Inhibition can be achieved when the activity value of a polypeptide or polynucleotide of the present invention relative to the control is less than about 80%, optionally less than about 50% (e.g., less than about 25-1%). Activation can be achieved when the activity value of a polypeptide or polynucleotide of the present invention relative to the control is greater than about 110%, optionally greater than about 150% (e.g., greater than about 200-500%, 1000-3000%, etc.).

The term “test compound,” “drug candidate,” “modulator,” or grammatical equivalents as used herein describes any molecule, either naturally-occurring or synthetic, e.g., protein, oligopeptide (e.g., from about 5-25 amino acids in length, preferably from about 10-20 or about 12-18 amino acids in length, preferably about 12, 15, or 18 amino acids in length), small organic molecule, polysaccharide, lipid, fatty acid, polynucleotide (e.g., plasmid), RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound” with some desirable property or activity, e.g., stimulating or inhibiting activity, creating variants of the lead compound, and evaluating the property and activity of those variant compounds. Often, high throughput screening (HTS) methods are employed for such an analysis.

A “small organic molecule” refers to an organic molecule, either naturally-occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

An “siRNA” or “RNAi” refers to a nucleic acid that forms a double-stranded RNA, which double-stranded RNA has the ability to reduce or inhibit expression of a gene or target gene when the siRNA expressed in the same cell as the gene or target gene. siRNA or RNAi thus refers to the double-stranded RNA formed by the complementary strands. The complementary portions of the siRNA that hybridize to form the double-stranded molecule typically have substantial or complete identity. In one embodiment, an siRNA refers to a nucleic acid that has substantial or complete identity to a target gene and forms a double-stranded siRNA. Typically, the siRNA is at least about 15-50 nucleotides in length (e.g., each complementary sequence of the double-stranded siRNA is about 15-50 nucleotides in length), and the double-stranded siRNA is about 15-50 base pairs in length, preferably about 20-30, about 20-25, or about 24-29 base pairs in length, e.g., 20, 21, 22, 23, 24, 25, 26, 27, 28, 29, or 30 base pairs in length.

“Determining the functional effect” refers to assaying for a compound that increases or decreases a parameter that is indirectly or directly under the influence of a polynucleotide or polypeptide of the present invention, e.g., measuring physical and chemical or phenotypic effects. Such functional effects can be measured by any means known to those skilled in the art, e.g., measuring changes in spectroscopic (e.g., fluorescence, absorbance, refractive index, etc.), hydrodynamic (e.g., shape, etc), chromatographic, or solubility properties for the protein; measuring inducible markers or transcriptional activation of the protein; measuring binding activity or binding assays, e.g., binding to antibodies, binding to proteins, binding to DNA; measuring changes in ligand binding affinity; measurement of calcium influx; measuring the accumulation of an enzymatic product of a polypeptide of the present invention or depletion of a substrate; measuring changes in enzymatic activity, e.g., kinase activity; measuring changes in protein levels of a polypeptide of the present invention; measuring RNA stability; measuring G-protein binding; measuring GPCR phosphorylation or dephosphorylation; measuring signal transduction, e.g., receptor-ligand interactions, second messenger concentrations (e.g., cAMP, IP3, or intracellular Ca²⁺, and the like); identifying downstream or reporter gene expression (e.g., CAT, luciferase, β-gal, GFP, and the like), e.g., via chemiluminescence, fluorescence, calorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably about 50%, and more preferably about 25-0%. Activation is achieved when the activity value relative to the control (untreated with activators) is about 110%, preferably about 150%, more preferably about 200-500% (i.e., two to five fold higher relative to the control), even more preferably at least about 1000-3000%.

The term “biological sample” includes sections of tissues such as biopsy and autopsy samples, and frozen sections taken for histological purposes. Such samples include blood and blood fractions or products (e.g., serum, plasma, platelets, red blood cells, and the like), sputum, tissue, cultured cells (e.g., primary cultures, explants, and transformed cells), stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., kidney, bladder, prostate, lymph node, liver, bone marrow, blood cell, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably about 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithm with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is about 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from about 20 to about 600, usually from about 50 to about 200, more usually from about 100 to about 150, in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithms that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the present invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally-occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of Smac/DIABLO. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′-end or at the 3′-end. Polypeptides can be truncated at the N-terminal end or the C-terminal end. Truncated versions of nucleic acid or polypeptide sequences can be naturally-occurring or recombinantly created.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally-occurring amino acid, as well as to naturally-occurring amino acid polymers and non-naturally occurring amino acid polymers.

The term “amino acid” refers to naturally occurring and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, and methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid.

Amino acids may be referred to herein by either their commonly known three-letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill in the art will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant,” when used with reference, e.g., to a cell, nucleic acid, protein, or vector, indicates that the cell, nucleic acid, protein, or vector has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all.

The term “heterologous,” when used with reference to portions of a nucleic acid, indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and may be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably at least ten times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill in the art will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and about 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of about 90-95° C. for about 30 sec-2 min., an annealing phase lasting about 30 sec-2 min., and an extension phase of about 72° C. for about 1-2 min. Protocols and guidelines for low and high stringency amplification reactions are provided, e.g., in Innis et al. (1990) PCR Protocols, A Guide to Methods and Applications, Academic Press, Inc. N.Y.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to about 110 or more amino acids primarily responsible for antigen recognition. The terms variable light chain (V_{L}) and variable heavy chain (V_{H}) refer to these light and heavy chains, respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology (Paul ed., 3d ed. 1993). While various antibody fragments are defined in terms of the digestion of an intact antibody, one of skill in the art will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler and Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow and Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to the polypeptides of the present invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg and Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced, or exchanged so that the antigen binding site (variable region) is linked to a constant region of a different or altered class, effector function, and/or species, or an entirely different molecule which confers new properties to the chimeric antibody, e.g., an enzyme, toxin, hormone, growth factor, drug, etc.; or (b) the variable region, or a portion thereof, is altered, replaced, or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect, the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least about two, three, four, or more times the background, and more typically more than at least about 10 to about 100 times the background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988) for a description of immunoassay formats and conditions that can be used to determine specific immunoreactivity).

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, and individual isomers are all intended to be encompassed within the scope of the present invention.

### III. Diagnostic and Prognostic Methods

The present invention also provides methods of diagnosing or providing a prognosis for a cancer, e.g., a cancer that underexpresses Smac/DIABLO. As used herein, the term “providing a prognosis” refers to providing a prediction of the probable course and outcome of a cancer or the likelihood of recovery from the cancer. In certain instances, cancer patients with positive Smac/DIABLO expression have a longer disease-specific survival as compared to those with negative Smac/DIABLO expression (see, Example 1). As such, the level of Smac/DIABLO expression can be used as a prognostic indicator, with positive expression as an indication of a good prognosis, e.g., a longer disease-specific survival.

The methods of the present invention can also be useful for diagnosing the severity of a cancer, e.g., a cancer that underexpresses Smac/DIABLO. As a non-limiting example, the level of Smac/DIABLO expression can be used to determine the stage or grade of a cancer such as renal cell carcinoma (RCC), e.g., according to the TNM system of classification (International Union Against Cancer, 6th edition, 2002). In certain instances, cancer patients with negative Smac/DIABLO expression have a more severe stage or grade of that type of cancer (see, Example 1). As such, the level of Smac/DIABLO expression can be used as a diagnostic indicator of the severity of a cancer or of the risk of developing a more severe stage or grade of the cancer.

Diagnosis or prognosis can involve determining the level of Smac/DIABLO expression (i.e., transcription or translation) or Smac/DIABLO intracellular localization in a patient and then comparing the level or localization to a baseline or range. Typically, the baseline value is representative of Smac/DIABLO expression levels or Smac/DIABLO intracellular localization in a healthy person not suffering from cancer. Variation of levels of a polypeptide or polynucleotide of the present invention from the baseline range (i.e., either up or down) indicates that the patient has a cancer or is at risk of developing a cancer. In some embodiments, the level of Smac/DIABLO expression or Smac/DIABLO intracellular localization is measured by taking a blood, urine, or tissue sample from a patient and measuring the amount of a polypeptide or polynucleotide of the present invention in the sample using any number of detection methods, such as those discussed herein.

Antibodies can be used in assays to detect differential protein expression and protein localization in patient samples, e.g., ELISA assays, immunoprecipitation assays, and immunohistochemical assays. In one embodiment, tumor tissue samples are used in immunohistochemical assays and scored according to standard methods known in the art. PCR assays can be used to detect expression levels of nucleic acids, as well as to discriminate between variants in genomic structure, such as insertion/deletion mutations, truncations, or splice variants. Immunohistochemistry and/or immunofluorescence techniques can be used to detect intracellular localization of Smac/DIABLO proteins.

In some embodiments, underexpression of Smac/DIABLO in a cancerous or potentially cancerous tissue in a patient may be diagnosed or otherwise evaluated by visualizing expression levels and localization in situ of a Smac/DIABLO polynucleotide, a Smac/DIABLO polypeptide, or fragments of thereof. Those skilled in the art of visualizing the presence or expression of molecules including nucleic acids, polypeptides, and other biological molecules in the tissues of living patients will appreciate that the gene expression information described herein may be utilized in the context of a variety of visualization methods. Such methods include, but are not limited to, single-photon emission-computed tomography (SPECT) and positron-emitting tomography (PET) methods. See, e.g., Vassaux and Groot-wassink, “In Vivo Noninvasive Imaging for Gene Therapy,” J. Biomedicine and Biotechnology, 2: 92-101 (2003).

PET and SPECT imaging shows the chemical functioning of organs and tissues, while other imaging techniques such as X-ray, CT, and MRI show structure. The use of PET and SPECT imaging is useful for qualifying and monitoring the development of cancers that underexpress Smac/DIABLO and/or therapy resistant cancers, including renal cancer (i.e., renal cell carcinoma), bladder cancer, prostate cancer, ovarian cancer, lung cancer, breast cancer, colon cancer, leukemias, B-cell lymphomas, myelomas and hepatocarcinomas. In some instances, the use of PET or SPECT imaging allows diseases to be detected years earlier than the onset of symptoms. The use of small molecules for labeling and visualizing the presence or expression of polypeptides and polynucleotides has had success, for example, in visualizing proteins in the brains of Alzheimer's patients, as described by, e.g., Herholz et al., Mol Imaging Biol., 6(4):239-69 (2004); Nordberg, Lancet Neurol., 3(9):519-27 (2004); Zakzanis et al., Neuropsychol Rev., 13(1):1-18 (2003); Kung et al, Brain Res., 1025(1-2):98-105 (2004); and Herholz, Ann Nucl Med., 17(2):79-89 (2003).

A Smac/DIABLO polypeptide, a Smac/DIABLO polynucleotide, or fragments thereof can be used in the context of PET and SPECT imaging applications. After modification with appropriate tracer residues for PET or SPECT applications, molecules which interact or bind with a Smac/DIABLO transcript or with any polypeptides encoded by those transcripts may be used to visualize the patterns of gene expression and facilitate diagnosis or prognosis of cancers that underexpress Smac/DIABLO.

### Iv. Assays for Modulators of Smac/DIABLO

Modulation of Smac/DIABLO, and corresponding modulation of cellular proliferation (e.g., tumor cell proliferation), can be assessed using a variety of in vitro and in vivo assays, including cell-based models. Such assays can be used to test for inhibitors and activators of Smac/DIABLO transcription or translation, or Smac/DIABLO protein activity, and consequently, inhibitors and activators of cellular proliferation, including modulators of chemotherapeutic and immunotherapeutic sensitivity and toxicity. Assays for modulation of Smac/DIABLO include cell-viability, cell proliferation, cell responses to apoptotic stimuli, gene transcription, mRNA arrays, kinase or phosphatase activity, interaction with other proteins, and the like. Such modulators of Smac/DIABLO are useful for treating diseases and disorders related to pathological cell proliferation, e.g., cancer, autoimmunity, aging, etc. Modulators of Smac/DIABLO activity can be tested using in vivo assays with cells expressing Smac/DIABLO or in vitro assays using either recombinant or naturally-occurring Smac/DIABLO protein, preferably human Smac/DIABLO. Wild-type Smac/DIABLO as well as truncated and alternatively spliced forms of Smac/DIABLO are also useful targets. The above-described assays can also be used to test for mimetics of Smac/DIABLO activity or function.

Measurement of cellular proliferation by modulation with Smac/DIABLO protein or Smac/DIABLO nucleic acid, either recombinant or naturally-occurring, can be performed using a variety of assays, e.g., in vitro, in vivo, and ex vivo, as described herein. A suitable physical, chemical, or phenotypic change that affects activity, e.g., enzymatic activity such as kinase activity, cell proliferation, or ligand binding can be used to assess the influence of a test compound on the polypeptide or polynucleotide of the present invention. When the functional effects are determined using intact cells or animals, one can also measure a variety of effects, such as ligand binding, DNA binding, kinase activity, transcriptional changes to both known and uncharacterized genetic markers (e.g., Northern blots), changes in cell metabolism, changes related to cellular proliferation, cell surface marker expression, DNA synthesis, marker and dye dilution assays (e.g., GFP and cell tracker assays), contact inhibition, tumor growth in nude mice, etc.

A. In Vitro Assays

Assays to identify compounds with Smac/DIABLO modulating activity can be performed in vitro. Such assays can use a full-length Smac/DIABLO protein, a variant thereof, a mutant thereof, a truncated form thereof, or a fragment thereof. Purified recombinant or naturally-occurring Smac/DIABLO protein can be used in the in vitro methods of the present invention. In addition to purified Smac/DIABLO protein, the recombinant or naturally-occurring Smac/DIABLO protein can be part of a cellular lysate or a cell membrane. As described below, the binding assay can either be solid state or soluble. Preferably, the protein or membrane is bound to a solid support, either covalently or non-covalently. Often, the in vitro assays of the present invention are substrate or ligand binding or affinity assays, and can be either non-competitive or competitive. Other in vitro assays include measuring changes in spectroscopic (e.g., fluorescence, absorbance, refractive index, etc.), hydrodynamic (e.g., shape, etc.), chromatographic, or solubility properties of the protein. Additional in vitro assays include enzymatic activity assays, such as phosphorylation or autophosphorylation assays.

In one embodiment, a high throughput binding assay is performed in which the Smac/DIABLO protein, a truncated form, or a fragment thereof is contacted with a potential modulator and incubated for a suitable amount of time. In certain instances, the potential modulator is bound to a solid support, and the Smac/DIABLO protein is added. In certain other instances, the Smac/DIABLO protein is bound to a solid support. A wide variety of modulators can be used, as described herein, including small organic molecules, peptides, polynucleotides, antibodies, and Smac/DIABLO binding proteins or nucleic acid analogs. A wide variety of assays can be used to identify Smac/DIABLO-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator.

In another embodiment, microtiter plates are first coated with either Smac/DIABLO protein or a Smac/DIABLO binding protein (e.g., anti-Smac/DIABLO antibody, inhibitor of apoptosis protein (IAP) family member, etc.), exposed to one or more test compounds, and then assayed for the ability of the one or more test compounds to potentiate the binding of Smac/DIABLO protein to the Smac/DIABLO binding protein. A labeled (e.g., fluorescent, enzymatic, radioactive isotope, etc.) binding partner of the coated protein, either Smac/DIABLO protein or Smac/DIABLO binding protein, is then exposed to the coated protein and test compounds. Unbound protein can be washed away as necessary in between exposures to Smac/DIABLO protein, a Smac/DIABLO binding protein, or a test compound. The presence of a detectable signal (e.g., fluorescence, colorimetric, radioactivity, etc.) greater than a control sample that was not exposed to a test compound indicates that the test compound potentiated the binding interaction between Smac/DIABLO protein and a Smac/DIABLO binding protein. One can also use chromatographic techniques such as high pressure liquid chromatography (HPLC) to evaluate elution profiles of Smac/DIABLO protein alone and Smac/DIABLO protein complexed to a Smac/DIABLO binding protein. In some embodiments, the binding partner is unlabeled, but exposed to a labeled antibody that specifically binds the binding partner.

B. Cell-Based In Vivo Assays

In another embodiment, Smac/DIABLO protein is expressed in a cell, and functional, e.g., physical, chemical, or phenotypic, changes are assayed to identify and modulators of cellular proliferation, e.g., tumor cell proliferation. Cells expressing Smac/DIABLO protein can also be used in binding assays and enzymatic assays. Preferably, the cells overexpress or underexpress Smac/DIABLO in comparison to a normal cell of the same type. Any suitable functional effect can be measured, as described herein. For example, cellular morphology (e.g., cell volume, nuclear volume, cell perimeter, and nuclear perimeter), ligand binding, kinase activity, apoptosis, cell surface marker expression, cellular proliferation, cellular localization of Smac/DIABLO protein or transcript, GFP positively and dye dilution assays (e.g., cell tracker assays with dyes that bind to cell membranes), DNA synthesis assays (e.g., ³H-thymidine and fluorescent DNA-binding dyes such as BrdU or Hoechst dye with FACS analysis), are all suitable assays to identify potential modulators using a cell-based system. Reporter gene assays are also useful in the present invention. Suitable cells for such cell-based assays include both primary cancer or tumor cells and cell lines as described herein, e.g., NC65, ACHN, and Caki-1 human RCC cell lines, A549 (lung), MCF₇ (breast, p53 wild-type), H1299 (lung, p53 null), Hela (cervical), PC3 (prostate, p53 mutant), and MDA-MB-231 (breast, p53 wild-type). Variants derived from these cell lines with specific gene modifications can also be used. Cancer cell lines can be p53 mutant, p53 null, or express wild-type p53. The Smac/DIABLO protein can be naturally-occurring or recombinant. Also, truncated forms or fragments of Smac/DIABLO or chimeric Smac/DIABLO proteins can be used in cell-based assays.

Cellular Smac/DIABLO polypeptide levels can be determined by measuring the level of Smac/DIABLO protein or mRNA. The level of Smac/DIABLO protein or proteins related to Smac/DIABLO are measured using immunoassays such as Western blotting, ELISA, immunofluorescence, and the like with an antibody that selectively binds to the Smac/DIABLO polypeptide or a fragment thereof. For measurement of mRNA, amplification, e.g., using PCR, RT-PCR, LCR, or hybridization assays, e.g., Northern hybridization, RNAse protection, and dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein. It is also useful to observe Smac/DIABLO protein translocation into and/or out of the mitochondria and other cellular compartments by, for example, confocal microscopy. Smac/DIABLO interaction with other proteins, including IAP family members, can be measured using standard immunoprecipitation and immunoblotting techniques. Smac/DIABLO binding to other factors, either DNA or protein, can be evaluated by labeling Smac/DIABLO protein, for example, with a fluorochrome.

C. Animal Models

Animal models of cellular proliferation also find use in screening for modulators of cellular proliferation. Similarly, transgenic animal technology including gene knockout technology, for example, as a result of homologous recombination with an appropriate gene targeting vector, or gene overexpression, will result in the absence or increased expression of Smac/DIABLO protein. The same technology can also be applied to make knockout cells. If desired, transgenic animals can be generated that possess tissue-specific expression or knockout of Smac/DIABLO protein. Preferably, transgenic animals are generated that overexpress Smac/DIABLO protein. Transgenic animals generated by such methods find use as animal models of cellular proliferation and are additionally useful in screening for modulators of cellular proliferation.

Knockout cells and transgenic mice can be made by insertion of a marker gene or other heterologous gene into an endogenous Smac/DIABLO gene site in the mouse genome via homologous recombination. Such mice can also be made by substituting an endogenous Smac/DIABLO with a mutated version of the Smac/DIABLO gene, or by mutating an endogenous Smac/DIABLO gene, e.g., by exposure to carcinogens. Transgenic mice and cells overexpressing Smac/DIABLO can be made introducing additional copies of the Smac/DIABLO gene in the mouse genome.

Typically, a DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice, it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988), Teratocarcinomas and Embryonic Stem Cells: A Practical Approach, Robertson, ed., IRL Press, Washington, D.C., (1987), and Pinkert, Transgenic Animal Technology: A Laboratory Handbook, Academic Press (2003).

D. Exemplary Assays

1. Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate a normal phenotype and require a solid substrate to attach and grow.

Soft agar growth or colony formation in suspension assays can be used to identify Smac/DIABLO modulators. Typically, transformed host cells (e.g., cells that grow on soft agar) are used in this assay. For example, RKO or HCT116 cell lines can be used. Techniques for soft agar growth or colony formation in suspension assays are described in Freshney, Culture of Animal Cells a Manual of Basic Technique, 3^{rd} ed., Wiley-Liss, New York (1994). See also, the methods section of Garkavtsev et al. (1996), supra.

2. Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, a labeling index with [³H]-thymidine at saturation density can be used to measure density limitation of growth. See, Freshney, supra. The transformed cells, when contacted with cellular proliferation modulators, regenerate a normal phenotype and become contact inhibited and would grow to a lower density.

Contact inhibition and density limitation of growth assays can be used to identify Smac/DIABLO modulators that are capable of inhibiting abnormal proliferation and transformation in host cells. Typically, transformed host cells (e.g., cells that are not contact inhibited) are used in this assay. For example, RKO or HCT116 cell lines can be used. In this assay, a labeling index with [³H]-thymidine at saturation density is a preferred method of measuring density limitation of growth. Transformed host cells are contacted with a potential Smac/DIABLO modulator and are grown for about 24 hours at saturation density in non-limiting medium conditions. The percentage of cells labeled with [³H]-thymidine is determined autoradiographically. See, Freshney, supra. The host cells contacted with a Smac/DIABLO modulator would give rise to a lower labeling index compared to control (e.g., transformed host cells transfected with a vector lacking an insert).

3. Growth Factor or Serum Dependence

Growth factor or serum dependence can be used as an assay to identify Smac/DIABLO modulators. Transformed cells have a lower serum dependence than their normal counterparts (see, e.g., Temin, J. Natl. Cancer Insti., 37:167-175 (1966); Eagle et al., J. Exp. Med., 131:836-879 (1970)); Freshney, supra. This is in part due to release of various growth factors by the transformed cells. When transformed cells are contacted with a Smac/DIABLO modulator, the cells would reacquire serum dependence and would release growth factors at a lower level.

4. Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, tumor angiogenesis factor (TAF) is released at a higher level in tumor cells than their normal counterparts. See, e.g., Folkman, Angiogenesis and cancer, Sem. Cancer Biol. (1992)). Other exemplified tumor specific markers include growth factors and cytokines.

Tumor specific markers can be assayed to identify Smac/DIABLO modulators which decrease the level of release of these markers from host cells. Typically, transformed or tumorigenic host cells are used. Various techniques which measure the release of these factors are described in, e.g., Freshney, supra. See also, Unkless et al., J. Biol. Chem. 249:4295-4305 (1974); Strickland and Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney, Anticancer Res. 5:111-130 (1985).

5. Invasiveness into Matrigel

The degree of invasiveness into Matrigel or some other extracellular matrix constituent can be used as an assay to identify Smac/DIABLO modulators which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Therefore, Smac/DIABLO modulators can be identified by measuring changes in the level of invasiveness between the host cells before and after the introduction of potential modulators. If a compound modulates Smac/DIABLO, its introduction into tumorigenic host cells would affect invasiveness.

Techniques described in Freshney, supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish.

6. G₀/G₁ Cell Cycle Arrest Analysis

G₀/G₁ cell cycle arrest can be used as an assay to identify Smac/DIABLO modulators. In this assay, cell lines such as RKO or HCT116 can be used to screen Smac/DIABLO modulators. The cells can be co-transfected with a construct comprising a marker gene, such as a gene that encodes green fluorescent protein, or a cell tracker dye. Methods known in the art can be used to measure the degree of G₁ cell cycle arrest. For example, a propidium iodide signal can be used as a measure for DNA content to determine cell cycle profiles on a flow cytometer. The percent of the cells in each cell cycle can be calculated. Cells contacted with a Smac/DIABLO modulator would exhibit, e.g., a higher number of cells that are arrested in G₀/G₁ phase compared to control.

7. Tumor Growth In Vivo

Effects of Smac/DIABLO modulators on cell growth can be tested in transgenic or immune-suppressed mice. Knockout transgenic mice can be made, in which the endogenous Smac/DIABLO gene is disrupted. Such knockout mice can be used to study effects of Smac/DIABLO, e.g., as a cancer model, as a means of assaying in vivo for compounds that modulate Smac/DIABLO, and to test the effects of restoring a wild-type or mutant Smac/DIABLO to a knockout mouse. Methods of generating knockout mice are described above.

Alternatively, various immune-suppressed or immune-deficient host animals can be used. For example, genetically athymic “nude” mouse (see, e.g., Giovanella et al., J. Natl. Cancer Inst. 52:921 (1974)), an SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportion of cases, while normal cells of similar origin will not. Hosts are treated with Smac/DIABLO modulators, e.g., by injection, optionally in combination with other cancer therapeutic agents, including chemotherapy, radiotherapy, immunotherapy and/or hormonal therapy. After a suitable length of time, preferably about 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, Smac/DIABLO modulators which are capable, e.g., of inhibiting abnormal cell proliferation or sensitizing tumor cells to cancer therapies, can be identified.

In immune-suppressed or immune-deficient host animals, the inoculating tumor cells preferably overexpress or underexpress Smac/DIABLO. The inoculating tumor cells are also preferably resistant to conventionally used cancer therapies. In one example, tumor cells resistant to death receptor-induced (e.g., DR5) apoptosis are inoculated as xenografts in SCID mice. The mice are subsequently treated with one or more Smac/DIABLO modulators (e.g., mimetics, agonists, etc.) combined with a death receptor agonist (e.g., a monoclonal antibody to DR5 or TRAIL).

Murine, rodent, and other animal tumor models for studying cancer are generally described, for example, in Immunodeficient Animals: Models for Cancer Research, Arnold et al., eds., 1996, S Karger Pub; Tumor Models in Cancer Research, Teicher, ed., 2002, Human Press; and Mouse Models of Cancer, Holland, ed., 2004, John Wiley & Sons. Specific murine tumor models for several different cancers have been described, including for example, metastatic colon cancer (Luo et al., Cancer Cell, 6:297 (2004)), breast cancer (Rahman and Sarkar, Cancer Res (2005) 65:364), cholangiocarcinoma (Chen et al., World J. Gastroenterol., (2005) 11:726), and prostate cancer (Tsingotjidou et al., Anticancer Res., 21:971 (2001) and U.S. Pat. No. 6,107,540).

### V. Screening Methods

The present invention also provides methods of identifying compounds that inhibit cancer growth or progression, for example, by increasing Smac/DIABLO protein and/or mRNA expression or potentiating the binding of Smac/DIABLO protein to a Smac/DIABLO binding protein such as an inhibitor of apoptosis protein (IAP) family member. The compounds find use in inhibiting the growth of and promoting the regression of a tumor that underexpresses Smac/DIABLO protein, for example, renal cancer (i.e., renal cell carcinoma), bladder cancer, prostate cancer, ovarian cancer, lung cancer, breast cancer, colon cancer, leukemias, B-cell lymphomas (e.g., non-Hodgkin's lymphomas, including Burkitt's, Small Cell, and Large Cell lymphomas), hepatocarcinoma, and multiple myeloma. The identified compounds can inhibit cancer growth or progression alone, or when used in combination with other cancer therapies, including chemotherapies, radiation therapies, hormonal therapies, immunotherapies, and combinations thereof.

Using the assays described herein, one can identify lead compounds that are suitable for further testing to identify those that are therapeutically effective modulating agents by screening a variety of compounds and mixtures of compounds for their ability to increase Smac/DIABLO protein and/or mRNA expression or potentiate the binding of Smac/DIABLO protein to a Smac/DIABLO binding protein. Compounds of interest can be either synthetic or naturally-occurring.

Screening assays can be carried out in vitro or in vivo. Typically, initial screening assays are carried out in vitro, and can be confirmed in vivo using cell based assays or animal models. For instance, compounds that increase Smac/DIABLO protein and/or mRNA expression or potentiate the binding of Smac/DIABLO protein to a Smac/DIABLO binding protein can promote cellular apoptosis resulting from the increased expression or binding interaction in comparison to cells unexposed to the test compound.

The screening methods are designed to screen large chemical or polymer (e.g., inhibitory RNA, including siRNA and antisense RNA, peptides, polynucleotides, small organic molecules, etc.) libraries by automating the assay steps and providing compounds from any convenient source to the assays, which are typically run in parallel (e.g., in microtiter formats on microtiter plates in robotic assays).

The present invention also provides in vitro assays in a high throughput format. For each of the assay formats described, “no modulator” control reactions, which do not include a modulator, provide, e.g., a background level of a Smac/DIABLO binding interaction to a Smac/DIABLO binding protein. In the high throughput assays of the present invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000-1,000,000 different compounds is possible using the integrated systems of the present invention. The steps of labeling, addition of reagents, fluid changes, and detection are compatible with full automation, for instance, using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

Essentially, any chemical compound can be tested as a potential modulator of Smac/DIABLO binding to a Smac/DIABLO binding protein for use in the methods of the present invention. Most preferred are generally compounds that can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

Compounds also include those that can regulate Smac/DIABLO transcription and/or post-transcriptional processing. Reporter systems can be used for this analysis.

In one preferred embodiment, modulators of Smac/DIABLO protein binding to a Smac/DIABLO binding protein are identified by screening a combinatorial library containing a large number of potential therapeutic compounds (potential modulator compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art (see, for example, Beeler et al., Curr Opin Chem. Biol. 9:277 (2005) and Shang and Tan, Curr Opin Chem. Biol. 9:248 (2005). Libraries of use in the present invention can be composed of amino acid compounds, nucleic acid compounds, carbohydrates, or small organic compounds. Carbohydrate libraries have been described in, for example, Liang et al., Science, 274:1520-1522 (1996) and U.S. Pat. No. 5,593,853.

Representative amino acid compound libraries include, but are not limited to, peptide libraries (see, e.g., U.S. Pat. Nos. 5,010,175; 6,828,422 and 6,844,161; Furka, Int. J. Pept. Prot. Res. 37:487-493 (1991); Houghton et al., Nature 354:84-88 (1991); and Eichler, Comb Chem High Throughput Screen. 8:135 (2005)), peptoids (PCT Publication No. WO 91/19735), encoded peptides (PCT Publication WO 93/20242), random bio-oligomers (PCT Publication No. WO 92/00091), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT/US96/10287; and Vaughn et al., Nature Biotechnology, 14(3):309-314 (1996)), and peptidyl phosphonates (Campbell et al., J. Org. Chem. 59:658 (1994)).

Representative nucleic acid compound libraries include, but are not limited to, genomic DNA, cDNA, mRNA, inhibitory RNA (e.g., RNAi, siRNA), and antisense RNA libraries. See, Ausubel, Current Protocols in Molecular Biology, supra, and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference or siRNA libraries, are reviewed in, for example, Downward, Cell 121:813 (2005) and Akashi, et al., Nat Rev Mol Cell Biol. 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA 90:6909-6913 (1993)); analogous organic syntheses of small compound libraries (Chen et al., J. Amer. Chem. Soc. 116:2661 (1994)); oligocarbamates (Cho et al., Science 261:1303 (1993)); benzodiazepines (e.g., U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

### VI. Gene Therapy

The present invention also provides methods of treating or inhibiting a cancer that underexpresses Smac/DIABLO or a therapy resistant cancer in a subject comprising administering to the subject a therapeutically effective amount of one or more Smac/DIABLO agonists such as nucleic acids encoding Smac/DIABLO, e.g., for gene therapy. As used herein, the term “gene therapy” refers to a therapeutic approach for introducing a specific polynucleotide into cells (e.g., cancer cells) to restore missing or abnormal gene expression; to increase reduced gene expression; to provide expression of a gene not typically expressed in the cells; or to inhibit gene expression. Examples of suitable gene therapy techniques include, without limitation, introducing wild-type copies of a gene into cancer cells that are missing expression of the gene or that have abnormal expression of the gene, inhibiting the expression of genes such as oncogenes in cancer cells, introducing genes into cancer cells that make them more vulnerable to cytotoxic therapy (e.g., chemotherapy, radiotherapy, immunotherapy, hormonal therapy, etc.), introducing genes into cancer cells that make them more easily detected and destroyed by the body's immune system, and inhibiting genes in cancer cells that are involved in angiogenesis.

In preferred embodiments of the present invention, the methods of treating or inhibiting a cancer involve administering a therapeutically effective amount of a Smac/DIABLO nucleic acid that restores missing Smac/DIABLO expression or increases reduced Smac/DIABLO expression in cancer cells. Without being bound to any particular theory, the introduction of Smac/DIABLO nucleic acid into cancer cells potentiates the effect of other cancer therapies by sensitizing the cells to such cytotoxic therapies. As a result, therapy resistant cancers can be effectively treated with gene therapy using Smac/DIABLO nucleic acid.

A variety of techniques are available for delivering the nucleic acid into cells for gene therapy including, but not limited to, in vivo and ex vivo techniques. For example, in vivo techniques can rely on the use of a virus (e.g., adenovirus) containing the desired nucleic acid sequence to be introduced into cancer cells. Alternatively, in vivo techniques can rely on the use of delivery systems that are complexed with or encapsulate the nucleic acid, e.g., lipoplexes or liposomal delivery systems containing plasmids, siRNA, antisense RNA, etc. One skilled in the art will also appreciate that the nucleic acid can be administered as a naked molecule, e.g., injected directly into the tumor. Ex vivo techniques involve removing cells from a patient, introducing the desired nucleic acid sequence into the cells, and placing the cells back into the patient. Suitable cells include cancer cells as well as cells of the immune system (e.g., to stimulate an immune response to the cancer cells). For example, cancer cells that have been removed and genetically altered can be injected back into the patient in hopes that immune cells will destroy them and any other cancer cells that resemble them. This approach may be useful in making the cancer cells more visible to the immune system, which often has a difficult time finding and attacking cancer cells in the body. Cells of the immune system such as dendritic cells can also be removed and genetically altered to make them more likely to attack cancer cells once they are put back into the body.

Numerous techniques are known in the art for the introduction of foreign genes into cells and may be used to construct the recombinant cells for purposes of gene therapy. Techniques which may be used include, but are not limited to, cell fusion, chromosome-mediated gene transfer, micro cell-mediated gene transfer, transfection, transformation, transduction, electroporation, infection (e.g., recombinant DNA viruses, recombinant RNA viruses), spheroplast fusion, microinjection, DEAE dextran, calcium phosphate precipitation, liposomes, lysosome fusion, synthetic cationic lipids, use of a gene gun or a DNA vector transporter, etc. For various techniques for transformation or transfection of mammalian cells, see, e.g., Keown et al., Methods Enzymol 185:527-37 (1990); Sambrook et al., Molecular Cloning, A Laboratory Manual, Third Edition, Cold Spring Harbor Laboratory Press, N.Y. (2001).

### VII. Methods of Administration and Pharmaceutical Compositions

Molecules and compounds identified that modulate the expression and/or function of Smac/DIABLO are useful in treating cancers that underexpress Smac/DIABLO. Smac/DIABLO modulators (e.g., mimetics, agonists, antagonists, etc.) can be administered alone or co-administered in combination with conventional chemotherapy, radiotherapy, hormonal therapy, and/or immunotherapy.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can comprise: (a) liquid solutions, such as an effective amount of a packaged Smac/DIABLO modulator suspended in diluents, e.g., water, saline, or PEG 400; (b) capsules, sachets, or tablets, each containing a predetermined amount of a Smac/DIABLO modulator, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a Smac/DIABLO modulator in a flavor, e.g., sucrose, as well as pastilles comprising the modulator in an inert base, such as gelatin and glycerin or sucrose and acacia emulsions, gels, and the like containing, in addition to the modulator, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

Injection solutions and suspensions can be prepared from sterile powders, granules, and tablets of the kind previously described. Cells transduced by Smac/DIABLO nucleic acids for ex vivo therapy can also be administered intravenously or parenterally as described above.

The pharmaceutical preparation is preferably in unit dosage form. In such form the preparation is subdivided into unit doses containing appropriate quantities of the active component, e.g., a Smac/DIABLO modulator. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or more Smac/DIABLO mimetics or agonists, optionally in combination with one or more chemotherapeutic agents, in a sustained release formulation. Typically, the Smac/DIABLO mimetic or agonist is administered therapeutically as a sensitizing agent that increases the susceptibility of tumor cells to other cytotoxic cancer therapies, including chemotherapy, radiation therapy, immunotherapy, and hormonal therapy. In some embodiments, the Smac/DIABLO mimetic can be a compound that targets the XIAP/caspase-9 interaction site as described in Sun et al., J. Med. Chem., 47:4147-4150 (2004). In other embodiments, the Smac/DIABLO agonist can be a compound that increases the expression of Smac/DIABLO protein and/or mRNA.

In therapeutic use for the treatment of cancer, the compounds utilized in the pharmaceutical methods of the present invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention, should be sufficient to effect a beneficial therapeutic response in the patient over time. The size of the dose will also be determined by the existence, nature, and extent of any adverse side-effects that accompany the administration of a particular vector or transduced cell type in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals. Non-human mammals treated using the present methods include domesticated animals (e.g., canine, feline, murine, rodentia, lagomorpha, etc.) and agricultural animals (bovine, equine, ovine, porcine, etc).

### VIII. Compositions, Kits, and Integrated Systems

The present invention provides compositions, kits, and integrated systems for practicing the assays described herein using the polypeptides or polynucleotides described herein, antibodies specific for the polypeptides or polynucleotides described herein, etc.

In one embodiment, the present invention provides assay compositions for use in solid phase assays. Such compositions can include, for example, one or more polypeptides or polynucleotides of the present invention immobilized on a solid support, and a labeling reagent. In each case, the assay compositions can also include additional reagents that are desirable for hybridization. Modulators of expression or activity of the polypeptides or polynucleotides of the present invention can also be included in the assay compositions.

In another embodiment, the present invention provides kits for carrying out the therapeutic, diagnostic, and prognostic assays described herein. The kits typically include one or more probes that comprise an antibody or nucleic acid sequence that specifically binds to the polypeptides or polynucleotides of the present invention, and a label for detecting the presence of the probe. The kits can find use, for example, for measuring the levels of Smac/DIABLO protein or Smac/DIABLO transcripts, or for measuring Smac/DIABLO-binding activity to a target protein (e.g., an inhibitor of apoptosis protein (IAP)). The kits may also include several polynucleotide sequences encoding polypeptides of the present invention. Kits can include any of the compositions noted above, and optionally further include additional components such as instructions to practice a high-throughput method of assaying for an effect on expression of the genes encoding the polypeptides of the present invention, or on activity of the polypeptides of the present invention, one or more containers or compartments (e.g., to hold the probe, labels, or the like), a control modulator of the expression or activity of polypeptides of the present invention, a robotic armature for mixing kit components, or the like.

In yet another embodiment, the present invention provides integrated systems for high-throughput screening of potential modulators for an effect on the expression or activity of the polypeptides of the present invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety. A number of robotic fluid transfer systems are available or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments described herein, e.g., by digitizing the image and storing and analyzing the image on a computer. A variety of commercially available peripheral equipment and software is available for digitizing, storing, and analyzing a digitized video or digitized optical image, e.g., using PC (Intel x86 or Pentium chip-compatible DOS®, OS2® WINDOWS®, WINDOWS NT®, WINDOWS950, WINDOWS980, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the present invention are easily used for viewing any sample, e.g., by fluorescent or dark field microscopic techniques.

### IX. Examples

The following example is offered to illustrate, but not to limit, the claimed invention.

### Example 1

### Downregulation of Smac/DIABLO Expression in Renal Cell Carcinoma and its Prognostic Significance

Second mitochondria-derived activator of caspase/direct inhibitor of apoptosis-binding protein with low pI (Smac/DIABLO) was recently identified as a protein that is released from mitochondria in response to apoptotic stimuli and promotes apoptosis by antagonizing inhibitor of apoptosis proteins (IAPs). Furthermore, Smac/DIABLO plays an important regulatory role in the sensitization of cancer cells to both immune- and drug-induced apoptosis. However, little is known about the clinical significance of Smac/DIABLO in various cancers, including renal cell carcinoma (RCC).

This example illustrates that the expression of Smac/DIABLO was lower in RCC compared with the autologous normal kidney. Sixty-four (82%) of 78 of RCC expressed Smac/DIABLO, and 18% were negative, whereas 100% of normal kidney tissues were positive. In stage VIII RCC, 96% expressed Smac/DIABLO, whereas only 50% expressed Smac/DIABLO in stage III/IV. Smac/DIABLO expression inversely correlated with the grade of RCC. Patients with RCC expressing Smac/DIABLO had a longer postoperative disease-specific survival than those without Smac/DIABLO expression in the 5-year follow-up. Transfection with Smac/DIABLO cDNA enhanced tumor necrosis factor-related apoptosis-inducing ligand (TRAIL)-mediated and cisplatin-mediated cytotoxicity in RCC.

The present study demonstrates for the first time that Smac/DIABLO expression was downregulated in RCC and that no Smac/DIABLO expression in RCC predicted a worse prognosis. In addition, transfection with Smac/DIABLO sensitized RCC to TRAIL/cisplatin-induced apoptosis. These results indicate that Smac/DIABLO expression in RCC may be used as a prognostic parameter, and that enhancement of Smac/DIABLO expression in RCC may potentiate conventional and experimental cytotoxic cancer therapies such as immunotherapy and chemotherapy.

### Materials and Methods

### Patients

Surgical specimens were obtained from 78 patients with RCC. These patients were selected randomly for this study. They included 57 male and 21 female patients, ranging in age from 19 to 83 years. Histologic diagnosis revealed that 70, 7, and 1 patient had clear cell carcinoma, papillary RCC, and Bellini duct carcinoma, respectively. Their histologic classification and staging data, according to the TNM system of classification (International Union Against Cancer, 6th edition, 2002), were: T1 (n=54), T2 (n=8), T3 (n=12), T4 (n=4); N0 (n=74), N1 (n=1), N2 (n=3); M0 (n=67), M1 (n=11); Stage I (n=48), Stage II (n=6), Stage III (n=8), Stage IV (n=16), and G1 (n=8), G2 (n=48), G3 (n=22), respectively. Specimens of normal kidney were collected from the same 78 patients with RCC. The paired samples were histologically confirmed RCC and normal kidney. Tissue specimens were also obtained from 2 patients with oncocytoma. The specimens were stored frozen at −80° C. until use for the assay of Smac/DIABLO expression. This study was performed after approval by a local Human Investigations Committee. Informed consent was obtained from each patient.

### RCC Cell Lines

NC65, ACHN, and Caki-1 human RCC cell lines (Fogh, Natl. Cancer Inst. Monogr., 49:5-9 (1978); Mizutani et al., Cancer Res., 55:590-596 (1995)) were maintained in monolayers on plastic dishes in RPMI-1640 medium (Gibco, Bio-cult, Glasgow, Scotland, U.K.) supplemented with 25 mM HEPES (Gibco), 2 mM L-glutamine (Gibco), 1% non-essential amino acid (Gibco), 100 units/ml penicillin (Gibco), 100 mg/ml streptomycin (Gibco), and 10% heat-inactivated fetal bovine serum (Gibco), hereafter referred to as complete medium.

### Western Blot Analysis

The expression of Smac/DIABLO in nonfixed fresh frozen tissues was determined by Western blot analysis as described in, e.g., Mizutani et al., J. Urol., 168:2650-2654 (2002). 20 μg of the sample proteins was electrophoresed on 7.5% polyacrylamide gels in Tris-glycine buffer and transferred to nitrocellulose membranes. The membrane was blocked for 30 minutes in blocking buffer (5% skim milk in 1% Tween-PBS) and probed first with the anti-Smac/DIABLO antibody (Imgenex, San Diego, Calif.) for 1 hour. The membrane was washed and then incubated with peroxidase-conjugated goat anti-rabbit IgG and developed with the use of an enhanced chemiluminescence detection kit (Amersham Pharmacia Biotech, Piscataway, N.J.). The relative expression of Smac/DIABLO protein was determined with a chemiluminescence imaging system and quantified by image analysis (Gel Doc 2000; BIO-RAD, Osaka, Japan).

The NC65 cell line constitutively expressed Smac/DIABLO and was used as the internal standard to compare assays. All samples were analyzed at the same time. Repeated measurements yielded the same results. When Smac/DIABLO expression was not visually observed by the Western blot analysis, it was regarded as no or negative expression. In contrast, expression of Smac/DIABLO was regarded as positive expression, if a visual band was detected by Western blot analysis regardless of the variation of the levels of expression. Positive expression meant unambiguous visual detection of Smac/DIABLO protein band by chemiluminescence and did not refer to the level of Smac/DIABLO expression.

Transient Transfection of RCC Cells with Smac/DIABLO cDNA

Transient transfection of RCC cells with Smac/DIABLO cDNA was determined as described in, e.g., Ng et al., Mol. Cancer. Ther., 1:1051-1058 (2002). The transfection of RCC cell lines was performed with the pcDNA3.1 vector containing full-length Smac/DIABLO or an empty vector using the polycationic liposome reagent Lipofectamine 2000 (Invitrogen, Carlsbad, Calif.). The transfection was done according to the manufacturer's instructions. Overexpression of Smac/DIABLO was observed by this transfection procedure (Ng et al., supra).

### Reagents

Recombinant human tumor necrosis factor-related apoptosis-inducing ligand (TRAIL) was purchased from Peprotech (Rocky Hill, N.J.). Cisplatin was supplied by Nippon Kayaku Co. Ltd. (Tokyo, Japan).

### Cytotoxicity Assay

Microculture tetrazolium dye (MTT) assay was used to determine tumor cell lysis as described in, e.g., Mizutani et al., Clin. Cancer Res., 9:1453-1460 (2003); and Mizutani et al., Cancer, 100:723-731 (2004). Briefly, 100 μl of target cell suspension (2×10⁴ cells) was added to each well of 96 well flat-bottom microtiter plates (Corning Glass Works, Corning, N.Y.), and each plate was incubated for 24 hours at 37° C. in a humidified 5% CO₂ atmosphere. After incubation, the supernatants were aspirated and tumor cells were washed three times with RPMI medium, and 200 μl of drug solution or complete medium for control were distributed in the 96 well plates. Each plate was incubated for 24 hours at 37° C. Following incubation, 20 μl of MTT working solution (5 mg/ml; Sigma Chemical Co., St. Louis, Mo.) was added to each culture well and the cultures were incubated for 4 hours at 37° C. in a humidified 5% CO₂ atmosphere. The culture medium was removed from the wells and replaced with 100 μl of isopropanol (Sigma Chemical Co.) supplemented with 0.05 N HCl. The absorbance of each well was measured with a microculture plate reader (Immunoreader; Japan Intermed Co. Ltd., Tokyo, Japan) at 540 nm. The percent cytotoxicity was calculated by the following formula: % cytotoxicity=(1-[absorbance of experimental wells/absorbance of control wells])×100.

### Statistical Analysis

All determinations were made in triplicate. For statistical analysis, Student's t-test and a Chi-square test were used. Postoperative disease-specific survival was determined by the Kaplan-Meier method. The Cox-Mantel test was used to establish the statistical difference in survival between RCC patients with and without Smac/DIABLO expression. A p-value of 0.05 or less was considered significant.

### Results

### SMAC/DIABLO Expression in RCC Cell Lines, RCC, and Normal Kidneys

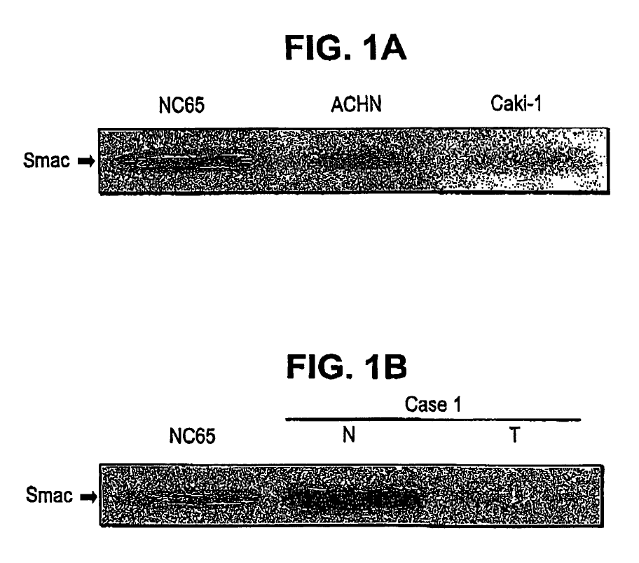

The levels of Smac/DIABLO in cell lysates of RCC cell lines, RCC, and normal kidneys were determined by Western blot analysis as described above. The NC65, ACHN, and Caki-1 RCC cell lines all expressed Smac/DIABLO, albeit at different levels (FIG. 1A). NC65 expressed the highest level of Smac/DIABLO and Caki-1 expressed the lowest level. The expression level of Smac/DIABLO in normal kidneys was higher than that in the NC65 line and the level of Smac/DIABLO expression in most RCCs was lower as represented in FIG. 1B.

Smac/DIABLO expression was determined in 78 normal kidneys and 78 RCCs. The percentages of cases expressing Smac/DIABLO and those not expressing Smac/DIABLO were determined and summarized in Table 1. Smac/DIABLO expression was detected in all normal kidney specimens. The Smac/DIABLO expression in normal kidneys in patients with RCC was similar to that in patients with renal pelvic cancer or ureteral cancer. Overall, 64 (82%) RCCs were positive for Smac/DIABLO and 14 (18%) were negative. The ratio of Smac/DIABLO expression in RCC compared to normal kidney was 0.27+0.03. In stage VIII RCC (n=54), 52 (96%) were positive and 2 (4%) were negative. However, in stage III/IV RCC (n=24), 12 (50%) were positive and 12 (50%) were negative. The ratio of Smac/DIABLO expression in stage VIII RCC compared to normal kidney was 0.37, and that in stage III/IV was 0.04. These findings were corroborated with grades of RCC. In grade ½ RCC (n=56), 53 (95%) were positive and 3 (5%) were negative. In contrast, in grade 3 RCC (n=22), 11 (50%) were positive and 11 (50%) were negative. The ratio of Smac/DIABLO expression in grade ½ and grade 3 RCCs compared to normal kidney was 0.35 and 0.06, respectively. These data show significant decrease of Smac/DIABLO expression in RCC as compared to normal kidneys. Furthermore, Smac/DIABLO expression inversely correlated with the stage progression and the increase of the histologic grade of RCC.

TABLE 1
Smac/DIABLO expression in RCC and normal kidneys.
Smac/DIABLO expression (%)^{a}

| | | | | | | Ratio of | |
|---|---|---|---|---|---|---|---|
| | | | | | | Smac/DIABLO | |
| | | | | | | expression level | |
| | | | | | | compared with | |
| RCC and | Total | Positive | | Negative | | normal kidney | |
| normal kidney | No. | No. | % | No. | % | Mean | SE |
| Kidney^{b} | | | | | | | |
| Normal | 78 | 78 | 100 | 0 | 0 | | |
| RCC | 78 | 64 | 82 | 14 | 18 | 0.27 | 0.03 |
| Tumor stage^{b} | | | | | | | |
| Stage I/II RCC | 54 | 52 | 96 | 2 | 4 | 0.37 | 0.04 |
| Stage III/IV RCC | 24 | 12 | 50 | 12 | 50 | 0.04 | 0.02^{c} |
| Tumor grade^{b} | | | | | | | |
| Grade 1/2 RCC | 56 | 53 | 95 | 3 | 5 | 0.35 | 0.04 |
| Grade 3 RCC | 22 | 11 | 50 | 11 | 50 | 0.06 | 0.02^{d} |

^{a}Smac/DIABLO expression in RCC and normal kidney was examined by Western blot analysis as described above.
^{b}p < 0.05 by Chi-square test.
^{c}p < 0.05 vs. Stage I/II RCC.
^{d}p < 0.05 vs. Grade 1/2 RCC.

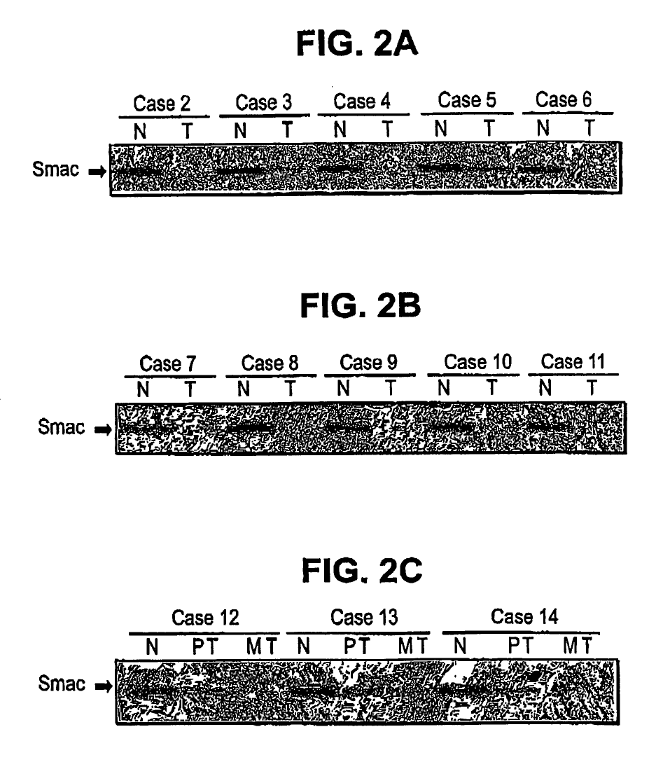

Representative data of Smac/DIABLO expression of RCC and normal kidneys from the same patients are shown in FIG. 1B and FIGS. 2A-C. The mean level of Smac/DIABLO expression in normal kidneys was approximately fourfold higher than that in RCCs. Smac/DIABLO expression was not seen in 14 out of 78 (18%) RCC (cases 2, 6-11). Experiments in 3 patients with metastatic RCC demonstrated that Smac/DIABLO expression was significantly lower in metastatic RCC than in primary RCC (FIG. 2C).

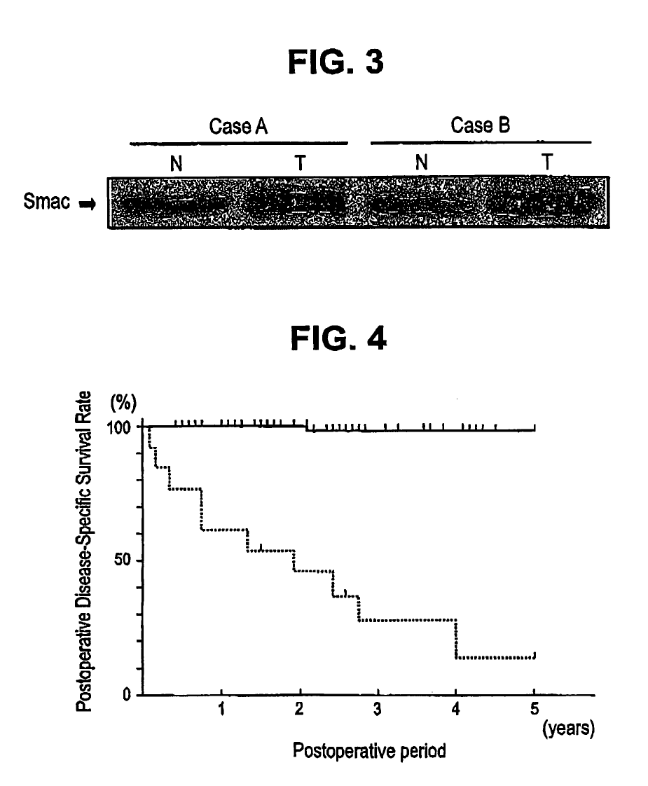

The level of Smac/DIABLO expression in clear-cell RCC was similar to that in papillary RCC. In contrast with RCC, Smac/DIABLO expression was upregulated in oncocytoma compared with normal kidney (FIG. 3).

These findings demonstrate that Smac/DIABLO expression was downregulated in RCC compared to normal kidneys and a significant population of patients with disease progression did not show Smac/DIABLO expression.

Correlation Between SMAC/DIABLO Expression and Postoperative Disease-Specific Survival in Patients with RCC

RCC patients undergoing radical nephrectomy were evaluated for the postoperative clinical course. Postoperative disease-specific survival was estimated by Kaplan-Meier analysis. Based on this analysis, patients with RCC were divided into two groups, namely, those with positive Smac/DIABLO expression and those with negative expression as described above. Patients with RCC with positive Smac/DIABLO expression had a longer disease-specific survival, compared to those with negative expression in the 5-year follow-up (FIG. 4). Moreover, it is noteworthy that only one patient with RCC with positive Smac/DIABLO expression died in this study, and the expression of Smac/DIABLO was very low in the primary tumor and negative in the metastatic tumor (representative case 14, FIG. 2C). These findings indicate that the level of Smac/DIABLO expression in RCC can be a prognostic indicator, and that positive Smac/DIABLO expression in RCC can be a good prognostic sign.

Sensitization of RCC Cells to Trail/Cisplatin-Mediated Cytotoxicity by Smac/DIABLO Transfection

Since Smac/DIABLO expression was downregulated in RCC, the effect of transfection of RCC with Smac/DIABLO cDNA on tumor growth and TRAIL/cisplatin-induced cytotoxicity was then examined. Cells transfected with pcDNA3.1-Smac/DIABLO have previously been demonstrated to overexpress the protein (Ng et al., supra). The transfection with Smac/DIABLO cDNA had no effect on the growth of NC65 and Caki-1 RCC cell lines. As shown in Table 2, transfection of NC65 cells with Smac/DIABLO enhanced TRAIL-mediated cytotoxicity. In addition, when the Caki-1 cell line that expressed less Smac/DIABLO compared with the NC65 line was used as a target, Smac/DIABLO transfection markedly potentiated TRAIL-induced cytotoxicity. Overexpression of Smac/DIABLO by transfection also sensitized NC65 cells to cisplatin-mediated cytotoxicity.

TABLE 2
Enhancement of the sensitivity of RCC cell lines to TRAIL/cisplatin
by transfection with Smac/DIABLO.

| | | % Cytotoxicity |
|---|---|---|
| RCC cell line | Treatment | (mean + S.D.)^{a} |
| NC65 | Transfection with control vector | 2.2 + 1.1 |
| NC65 | Transfection with pcDNA- | 0.0 + 1.9 |
| | Smac/DIABLO | |
| NC65 | Transfection with control vector + | 6.6 + 1.1 |
| | TRAIL (10 ng/ml) | |
| NC65 | Transfection with pcDNA- | 26.7 + 1.6^{b} |
| | Smac/DIABLO + TRAIL (10 ng/ml) | |
| NC65 | Transfection with control vector + | 17.8 + 3.3 |
| | cisplatin (10 mM) | |
| NC65 | Transfection with pcDNA- | 48.9 + 1.1^{c} |
| | Smac/DIABLO + cisplatin (10 mM) | |
| Caki-1 | Transfection with control vector | 2.4 + 1.0 |
| Caki-1 | Transfection with pcDNA- | 3.8 + 0.4 |
| | Smac/DIABLO | |
| Caki-1 | Transfection with control vector + | 14.1 + 1.1 |
| | TRAIL (10 ng/ml) | |
| Caki-1 | Transfection with pcDNA- | 50.1 + 2.9^{b} |
| | Smac/DIABLO + TRAIL (10 ng/ml) | |

^{a}The cytotoxic effect of transfection with control vector/pcDNA-Smac/DIABLO with or without TRAIL/cisplatin on NC65 and Caki-1 RCC cell lines was assessed by an 1-day MTT assay.
^{b}p < 0.05 vs. transfection with control vector + TRAIL.
^{c}p < 0.05 vs. transfection with control vector + cisplatin.

These findings indicate that low expression of Smac/DIABLO in RCC is associated with drug/immune resistance, and that overexpression of Smac/DIABLO enhances TRAIL/cisplatin-mediated apoptosis in RCC.

### Discussion

For the first time, evidence is presented that Smac/DIABLO expression was downregulated in RCC compared with autologous normal kidneys, and that the level of Smac/DIABLO expression inversely correlated with both the progression of the stage and the increase of the grade of RCC. Furthermore, this study shows that RCC patients with positive Smac/DIABLO expression had a longer disease-specific survival as compared to those with negative expression in the 5-year follow-up. These findings demonstrate that Smac/DIABLO in RCC plays an important role in regulating apoptosis and can be of prognostic value in RCC.

Patients with RCC respond very poorly to chemotherapy and radiotherapy (Yagoda, Semin. Urol., 7:199-206 (1989)). RCC cell lines have been described to be resistant to apoptosis inducing stimuli. A set of cell lines derived from human RCC almost completely lacked the expression of caspase 3 and further expressed other caspases at low levels (Kolenko et al., Cancer Res., 59:2838-2842 (1999)). Such alteration might contribute to RCC development. A recent study by Gerhard et al., Br. J. Cancer, 89:2147-2154 (2003) examined the functional competence of the apoptosome in RCC cell lines and RCC fresh tissues. They found that the apoptosome is structurally and functionally intact in both RCC cell lines and primary RCC by the criteria of adding exogenous cytochrome c. These findings suggested that the apoptosome may not be directly involved in resistance. Their study, however, did not examine the activation of the apoptosome and apoptosis by intrinsic cytochrome c and the role of Smac/DIABLO in the activation. The interaction of the apoptosome with low expression of cytochrome c or Smac/DIABLO may not be sufficient to trigger the apoptosome. The present study shows that low expression of Smac/DIABLO with a possibly intact apoptosome may be associated with resistance and illustrates the therapeutic effect of overexpressing Smac/DIABLO in the reversal of resistance.

The present study has shown that the expression of Smac/DIABLO in RCC was significantly lower than that in the normal kidney and approximately 20% RCC lacked Smac/DIABLO expression, although all normal kidney specimens expressed Smac/DIABLO. A recent study by Yoo et al., APMIS, 111:382-388 (2003) has reported analysis of archival tissues of carcinoma and sarcoma by immunohistochemical analysis for the expression of Smac/DIABLO. Smac/DIABLO expression was observed in 62% of carcinomas and 22% of sarcomas. The level of Smac/DIABLO expression varied depending on the individual tumor. For instance, 2 out of 10 prostate carcinomas were positive for Smac/DIABLO, whereas the remaining 8 were negative. Normal tissues adjacent to the cancer showed various degrees of Smac/DIABLO expression. However, in this report, there were no data on the expression of Smac/DIABLO in RCC.

This study is the first to demonstrate that Smac/DIABLO expression in RCC predicted the clinical outcome. Since Smac/DIABLO is a proapoptotic regulatory molecule, it is reasonable to assume that in spite of treatments, clones of cells which do not express Smac/DIABLO will not undergo apoptosis and will be selected to grow more easily and rapidly than clones that overexpress Smac/DIABLO. In addition, this study has shown that Smac/DIABLO was less expressed in the metastatic RCC than in the primary RCC. These findings indicate that Smac/DIABLO agonists may provide a therapeutic means of preventing metastasis and growth of RCC.

Cytotoxic chemotherapy, an integral part of the therapeutic approach for many solid tumors, has shown little or no antitumor activity against RCC and has played no role in either an adjuvant or a neoadjuvant support therapy (Yagoda, supra). Immunotherapy including interleukin-2 and interferon-α is relatively effective against metastatic RCC, and the overall response rate of immunotherapy and/or chemotherapy has gradually improved. However, the response rate is approximately 20%, and metastasis and recurrence still remain major problems in the therapy for RCC (Bukowski, Cancer, 80:1198-1220 (1997)). Therefore, new therapeutic approaches are required. The downregulation of Smac/DIABLO expression in RCC compared to the normal kidney identifies Smac/DIABLO as a molecular therapeutic target. The observation that overexpression of Smac/DIABLO in RCC by transfection resulted in high sensitivity to TRAIL/cisplatin-mediated killing is clinically relevant in the management of patients with RCC. The endogenous low level of Smac/DIABLO in RCC may not be adequate to neutralize the anti-apoptotic mechanism regulated by IAPs. Thus, immunotherapy and/or chemotherapy in combination with Smac/DIABLO agonists can be a useful therapeutic strategy against RCC. Furthermore, enhancement of Smac/DIABLO expression by gene therapy may also provide a novel therapeutic means of overcoming the resistance of RCC to immunotherapy and/or chemotherapy.

IAPs such as XIAP are highly expressed in various cancers and are associated with poor prognosis and resistance to apoptosis (Deveraux et al., Genes Dev., 13:239-252 (1999); Sasaki et al., Cancer Res., 60:5659-5666 (2000)). Expression of XIAP in RCC has been shown to be higher than that in the normal kidney. Since XIAP blocks apoptosis at the effector phase, strategies targeting XIAP, e.g., with Smac/DIABLO mimetics, can be especially effective at overcoming resistance to apoptosis. Smac/DIABLO is able to bind to IAP family members, and XIAP is a predominant Smac/DIABLO binding protein. Smac/DIABLO binds to XIAP, displaces XIAP from caspase-9, promotes cleavage of effector caspases, and induces apoptosis (Goyal, Cell, 104:805-808 (2001); Srinivasula et al., Nature, 410:112-116 (2001)). Therefore, in certain instances, the measurement of XIAP expression as well as Smac/DIABLO expression may be necessary for the accurate evaluation of the efficacy of therapy with Smac/DIABLO.

Drugs that can antagonize IAPs may have benefits particularly when combined with chemotherapeutic drugs or TRAIL. For instance, Arnt et al., J. Biol. Chem., 277:44236-44243 (2002) found that the first four amino acids of Smac/DIABLO increased apoptosis in cell lines treated with paclitaxel, etoposide, camptothecin, and doxorubicine.

Cancer therapy using TRAIL or anti-DR4/5 monoclonal antibody is currently being investigated in clinical trials due to their low toxicity to normal tissues (Ashkenazi et al., Science, 281:1305-1308 (1998); Walczak et al., Nature Med., 5:157-163 (1999)). However, not all tumors respond to TRAIL, and resistance to TRAIL has been shown to be overcome by drugs (Mizutani et al., Clin. Cancer Res., 5:2605-2612 (1999); Mizutani et al., Eur. J. Cancer, 38:167-176 (2002)), by overexpression of Smac/DIABLO, or by Smac/DIABLO peptides (Fulda et al., Nat. Med., 8:808-815 (2002); Ng et al., supra; Arnt et al., supra). Thus, analysis of the expression of Smac/DIABLO in cancer may be helpful for determining therapeutic modalities such as TRAIL therapy.

The findings of this study showed that patients with RCC with positive Smac/DIABLO expression had a longer disease-specific survival than those with negative expression. Fundamentally, patients without metastasis or recurrence received no postoperative treatments. The first- and the second-line treatments for metastasis or recurrence were intramuscular interferon-α monotherapy and combination therapy with intramuscular interferon-α and intravenous interleukin-2, respectively. The third-line treatment or surgery was dependent on each patient. Therefore, differing therapies may in part account for the different survival curves.

The dramatic post-operative disease-specific survival advantage for Smac/DIABLO-positive RCCs is the central issue in this study. In stage III/IV RCC patients (n=24), 10 (83%) patients with negative Smac/DIABLO expression (n=12) died of RCC. In contrast, only one (8%) patients with positive expression (n=12) died.

In conclusion, the present study demonstrated that Smac/DIABLO expression was downregulated in RCC, and that negative Smac/DIABLO expression was a poor prognostic sign. Furthermore, elevated Smac/DIABLO expression by transfection rendered resistant RCC cells sensitive to TRAIL/cisplatin-mediated cytotoxicity. These findings indicate that the assessment of Smac/DIABLO expression is particularly useful in the management of RCC. Since Smac/DIABLO expression can be used as a prognostic indicator in patients with RCC, the accurate prediction of prognosis can help select patients for more intensive surgical or immunochemotherapeutic approaches in combination with Smac/DIABLO agonists.

It is understood that the example and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing a cancer that underexpresses Smac/DIABLO, the method comprising the steps of:
(a) contacting a tissue sample with an antibody that specifically binds to Smac/DIABLO protein; and
(b) determining whether or not Smac/DIABLO protein is underexpressed in the sample, thereby diagnosing the cancer that underexpresses Smac/DIABLO.

2. The method of claim 1, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

3. The method of claim 1, wherein the tissue sample is a needle biopsy, a surgical biopsy, or a bone marrow biopsy.

4. The method of claim 3, wherein the tissue sample is at least one of fixed or embedded in paraffin.

5. The method of claim 1, wherein the antibody is a monoclonal antibody.

6. A method of diagnosing a cancer that underexpresses Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid in the sample is underexpressed in the sample, thereby diagnosing the cancer that underexpresses Smac/DIABLO.

7. The method of claim 6, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

8. The method of claim 6, wherein the tissue sample is a needle biopsy, a surgical biopsy, or a bone marrow biopsy.

9. The method of claim 6, wherein the first oligonucleotide comprises SEQ ID NO:1 and the second oligonucleotide comprises SEQ ID NO:2.

10. A method of providing a prognosis for a cancer that underexpresses Smac/DIABLO, the method comprising the steps of:
(a) contacting a tissue sample with an antibody that specifically binds to Smac/DIABLO protein; and
(b) determining whether or not Smac/DIABLO protein is underexpressed in the sample, thereby providing a prognosis for the cancer that underexpresses Smac/DIABLO.

11. The method of claim 10, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

12. The method of claim 10, wherein the tissue sample is a needle biopsy, a surgical biopsy, or a bone marrow biopsy.

13. The method of claim 10, wherein the tissue sample is a metastatic cancer tissue sample.

14. The method of claim 10, wherein the tissue sample is from kidney, bladder, prostate, ovary, bone, lymph node, or liver.

15. The method of claim 10, wherein the antibody is a monoclonal antibody.

16. A method of providing a prognosis for a cancer that underexpresses Smac/DIABLO, the method comprising the steps of:
(a) contacting a tissue sample with a primer set of a first oligonucleotide and a second oligonucleotide that each specifically hybridize to a Smac/DIABLO nucleic acid;
(b) amplifying the Smac/DIABLO nucleic acid in the sample; and
(c) determining whether or not the Smac/DIABLO nucleic acid is underexpressed in the sample, thereby providing a prognosis for the cancer that underexpresses Smac/DIABLO.

17. The method of claim 16, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

18. The method of claim 16, wherein the tissue sample is a needle biopsy, a surgical biopsy, or a bone marrow biopsy.

19. The method of claim 16, wherein the tissue sample is a metastatic cancer tissue sample.

20. The method of claim 16, wherein the tissue sample is from kidney, bladder, prostate, ovary, bone, lymph node, or liver.

21. The method of claim 16, wherein the first oligonucleotide comprises SEQ ID NO:1 and the second oligonucleotide comprises SEQ ID NO:2.

22. An isolated primer set, the primer set comprising a first oligonucleotide and a second oligonucleotide, the oligonucleotides comprising a nucleotide sequence of about 50 nucleotides or less; wherein the first oligonucleotide comprises SEQ ID NO:1 and the second oligonucleotide comprises SEQ ID NO:2.

23. A method of localizing a cancer that underexpresses Smac/DIABLO in vivo, the method comprising the step of imaging in a subject a cell underexpressing Smac/DIABLO, thereby localizing the cancer in vivo.

24. The method of claim 23, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

25. A method of identifying a compound that inhibits a cancer that underexpresses Smac/DIABLO, the method comprising the steps of:
(a) contacting a cell expressing Smac/DIABLO with a compound; and
(b) determining the effect of the compound on Smac/DIABLO expression, thereby identifying a compound that inhibits the cancer that underexpresses Smac/DIABLO.

26. The method of claim 25, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

27. The method of claim 25, wherein the compound increases Smac/DIABLO expression.

28. A method of identifying a compound that inhibits a therapy resistant cancer, the method comprising the steps of:
(a) contacting a cell expressing Smac/DIABLO with a compound; and
(b) determining the effect of the compound on Smac/DIABLO expression, thereby identifying a compound that inhibits the therapy resistant cancer.

29. The method of claim 28, wherein the therapy resistant cancer is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

30. The method of claim 28, wherein the compound increases Smac/DIABLO expression.

31. The method of claim 28, wherein the compound sensitizes the cell to apoptosis induced by cell signaling through a death receptor.

32. A method of treating or inhibiting a cancer that underexpresses Smac/DIABLO in a subject comprising administering to the subject a therapeutically effective amount of one or more Smac/DIABLO mimetics or agonists.

33. The method of claim 32, wherein the cancer that underexpresses Smac/DIABLO is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

34. The method of claim 32, wherein the Smac/DIABLO mimetic binds to one or more inhibitor of apoptosis proteins (IAPs).

35. The method of claim 32, wherein the Smac/DIABLO agonist is a Smac/DIABLO nucleic acid.

36. The method of claim 35, wherein the Smac/DIABLO nucleic acid increases Smac/DIABLO expression.

37. The method of claim 32, wherein the Smac/DIABLO agonist is co-administered with another cancer therapy.

38. A method of treating or inhibiting a therapy resistant cancer in a subject comprising administering to the subject a therapeutically effective amount of one or more Smac/DIABLO mimetics or agonists.

39. The method of claim 38, wherein the therapy resistant cancer is selected from the group consisting of renal cell carcinoma, bladder cancer, prostate cancer, ovarian cancer, breast cancer, colon cancer, lung cancer, leukemia, non-Hodgkin's lymphoma, multiple myeloma, and hepatocarcinoma.

40. The method of claim 38, wherein the Smac/DIABLO mimetic binds to one or more inhibitor of apoptosis proteins (IAPs).

41. The method of claim 38, wherein the Smac/DIABLO agonist is a Smac/DIABLO nucleic acid.

42. The method of claim 41, wherein the Smac/DIABLO nucleic acid increases Smac/DIABLO expression.

43. The method of claim 38, wherein the Smac/DIABLO agonist is co-administered with another cancer therapy.
